(19) United States
(12) Patent Application Publication
(10) Pub. No.: US 2025/0073219 A1
(43) Pub. Date: Mar. 6, 2025

(54) NEW COMPOSITION COMPRISING IMMUNOMODULATORY DRUG

(71) Applicant: NANEXA AB, Uppsala (SE)

(72) Inventors: Joel HELLRUP, Uppsala (SE); Jonas FRANSSON, Uppsala (SE); Mårten ROOTH, Uppsala (SE); Anders JOHANSSON, Uppsala (SE); David WESTBERG, Uppsala (SE); Erik LINDAHL, Uppsala (SE)

(21) Appl. No.: 18/567,452

(22) PCT Filed: Jun. 10, 2022
(86) PCT No.: PCT/GB2022/051464

§ 371 Date: Dec. 6, 2023

(30) Foreign Application Priority Data
Jun. 10, 2021 (EP) 21178675.1

(51) Int. Cl. A61K 31/454; A61K 9/50

(52) U.S. Cl. CPC A61K 31/454; A61K 9/501; A61K 9/5089

(57) ABSTRACT

The invention relates to a plurality of coated particles containing a solid core containing one of the compounds lenalidomide and pomalidomide, or a pharmaceutically acceptable salt thereof. The core has a mean diameter of the between 0.1 pm and 50 pm. The core is a coated by a coating surrounding, enclosing and/or encapsulating the core. The coating contains a mixed oxide selected from the group consisting of iron oxide, zinc oxide, silicon oxide, titanium oxide, and aluminium oxide.

### FIELD OF THE INVENTION

The present invention relates to new formulations for use in, for example, the field of drug delivery and in particular in the treatment of hematologic cancers, and in particular multiple myeloma as well as methods of production of said formulations.

### BACKGROUND TO THE INVENTION

The listing or discussion of an apparently prior-published document in this specification should not necessarily be taken as an acknowledgement that the document is part of the state of the art or common general knowledge.

Hematologic cancers, also referred to as plasma cell cancers or plasma cell neoplasms, are a group of disorders involving mature B-cells. These disorders result in the excessive production of specific monoclonal antibodies. The antibodies build up in the bone marrow and can cause the blood to thicken and/or damage the kidneys. This class of neoplasms includes monoclonal gammopathy of undetermined significance (MGUS), plasmacytoma and multiple myeloma, monoclonal immunoglobulin deposition diseases, plasma cell myeloma (PCM), plasma cell neoplasms with associated paraneoplastic syndromes.

Multiple myeloma is a type of cancer that affects plasma cells. The rapid reproduction of malignant myeloma cells in bone marrow eventually outweighs the production of healthy cells. As a result, the cancerous cells begin to accumulate in the bone marrow, displacing healthy white blood cells and red blood cells.

Multiple myeloma is considered treatable, but generally incurable. Remission may be brought about using steroids, chemotherapy, targeted therapy and/or stern cell transplants. Bisphosphonates and radiation therapy are sometimes used to reduce pain from bone lesions.

The cause of multiple myeloma is unknown, but risk factors for its development include obesity, radiation exposure, family history and/or exposure to certain chemicals. Multiple myeloma, as well as other plasma cell neoplasms, may cause a serious condition called amyloidosis, which involves the failure of peripheral nerves and organs.

Globally, multiple myeloma affected 488,000 people and resulted in 101,100 deaths in 2015. The onset is usually around the age of 60 and the condition is more common in men than women. Without treatment, typical survival is seven months.

The preferred treatment for patients under the age of 65 is high-dose chemotherapy, to be followed by a stem cell transplant. Current drug treatments include bortezomib (a proteasome inhibitor) and well as lenalidomide (an immunomodulator), which is usually coadministered along with the corticosteroid, dexamethasone.

However, patients over the age of 65 and patients with significant comorbidities often cannot tolerate stem-cell transplantation. For such patients, the standard of care is been chemotherapy using with the alkylation agent, melphalan, often coadministereted with the corticosteroid, prednisone and, more recently, with bortezomib.

The chemotherapeutic agent lenalidomide (3-(4-amino-1-3-dihydro-l-ovo-2H-isoindol-2yl)-2,6-piperidinedione) is a 4-amino-glutamyl analogue that belongs to a group of immunomodulatory compounds that includes pomalidomide, thalidomide, iberdomide, apremilast, and their analogues. These compounds are reported to have pleiotropic anti-myeloma properties including immune-modulation, anti-angiogenic, anti-inflammatory and antiproliferative effects. Lenalidomide is available in a peroral administration form, and the current standard-of-care treatment for multiple myeloma involves a daily oral dose of 25 mg (together with dexamethasone). After stem cell transplantation, patients are generally administered an oral daily dose of 10 mg.

For treatment of plasma cell neoplasms, including multiple myeloma, it would be advantageous to provide an extended release composition, in which active ingredient is released at a desired and predictable rate in vivo following administration (e.g. by way of injection), in order to ensure a more optimal pharmacokinetic profile.

In the case of any sustained release composition, it is of critical importance that the release profile of active ingredient shows minimal initial rapid release of active ingredient, that is a large concentration of drug in plasma shortly after administration. Such a ‘burst’ release will result in unwanted, high concentrations active ingredient, and may be hazardous in the case of drugs that have a narrow therapeutic window or are toxic at high plasma concentrations. In the case of lenalidomide, this is particularly problematic due to the drug's cytotoxicity and the prevelance of side effects, which commonly include diarrhoea, pruritis (itchiness), joint pain, fever, headache, and trouble sleeping, with more severe side effects including low blood platelet counts, which in turn can lead to thrombosis, and low white blood cell counts.

In the case of an injectable suspension of an active ingredient, it is also important that the size of the suspended particles is controlled so that they can be injected through a needle. If large, aggregated particles are present, they will not only block the needle, through which the suspension is to be injected, but also will not form a stable suspension within (i.e. they will instead tend to sink to the bottom of) the injection liquid.

Thus, there is a significant unmet clinical need to an provide improved, effective and more patient-friendly treatment of hematologic cancers, including multiple myeloma, which treatments may be longer lasting and/or more effective.

Atomic layer deposition (ALD) is a technique that is employed to deposit thin films comprising a variety of materials, including organic, biological, polymeric and, especially, inorganic materials, such as metal oxides, on solid substrates. It is an enabling technique for atomic and close-to-atomic scale manufacturing (ACSM) of materials, structures, devices and systems in versatile applications (see, for example, Zhang et al. Nanomanuf. Metrol. 2022, https://doi.org/10.1007/s41871-022-00136-8). Based on its self-limiting characteristics, ALD can achieve atomic-level thickness that is only controlled by adjusting the number of growth cycles. Moreover, multilayers can be deposited, and the properties of each layer can be customized at the atomic level.

Due to its atomic-level control, ALD is used as a key technique for the manufacturing of, for example, next-generation semiconductors, or in atomic-level synthesis of advanced catalysts as well as in the precise fabrication of nanostructures, nanoclusters, and single atoms (see, for example, Zhang et al. vide supra).

The technique is usually performed at low pressures and elevated temperatures. Film coatings are produced by alternating exposure of solid substrates within an ALD reactor chamber to vaporized reactants in the gas phase. Substrates can be silicon wafers, granular materials or small particles (e.g. microparticles or nanoparticles).

The coated substrate is protected from chemical reactions (decomposition) and physical changes by the solid coating. ALD can also potentially be used to control the rate of release of the substrate material within a solvent, which makes it of potential use in the formulation of active pharmaceutical ingredients.

In ALD, a first precursor, which can be metal-containing, is fed into an ALD reactor chamber (in a so called ‘precursor pulse’), and forms an adsorbed atomic or molecular monolayer at the surface of the substrate. Excess first precursor is then purged from the reactor, and then a second precursor, such as water, is pulsed into the reactor.

This reacts with the first precursor, resulting in the formation of a monolayer of e.g. metal oxide on the substrate surface. A subsequent purging pulse is followed by a further pulse of the first precursor, and thus the start of a new cycle of the same events (a so called ‘ALD cycle’).

The thickness of the film coating is controlled by inter alia the number of ALD cycles that are conducted.

In a normal ALD process, because only atomic or molecular monolayers are produced during any one cycle, no discernible physical interface is formed between these monolayers, which essentially become a continuum at the surface of the substrate.

In international patent application WO 2014/187995, a process is described in which a number of ALD cycles are performed, which is followed by periodically removing the resultant coated substrates from the reactor and conducting a re-dispersion/agitation step to present new surfaces available for precursor adsorption.

The agitation step is done primarily to solve a problem observed for nano- and microparticles, namely that, during the ALD coating process, aggregation of particles takes place, resulting in ‘pinholes’ being formed by contact points between such particles. The re-dispersion/agitation step was performed by placing the coated substrates in water and sonicating, which resulted in deagglomeration, and the breaking up of contact points between individual particles of coated active substance.

The particles were then loaded back into the reactor and the steps of ALD coating of the powder, and deagglomerating the powder were repeated 3 times, to a total of 4 series of cycles. This process has been found to allow for the formation of coated particles that are, to a large extent, free of pinholes (see also Hellrup et al, Int. J. Pharm., 529, 116 (2017)).

Objects of the invention include seeking to alleviate, at least in part, the drawbacks of the prior art. Objects of the invention thus include the provision of an improved drug delivery system for lenalidomide, for the related immunomodulatory compound pomalidomide as well as the provision of a depot or depot-forming formulations for either or both of said compounds, such as for example lenalidomide.

We have made a novel (e.g. injectable) composition comprising, in particular, lenalidomide, in which ALD is used to coat microparticles of active ingredient with a mixture of metal and/or metalloid oxide coating layers, which coated particles may then be suspended in an (e.g. aqueous) vehicle. This composition produces an advantageous pharmacokinetic profile by releasing active ingredient over an extended period of time to provide a therapeutically-effective level of drug in systemic circulation, without any significant initial burst effect. The present disclosure also provides a novel composition of lenalidomide or pomalidomide, in which ALD is used to coat microparticles of the compound with a specific oxide coating as described herein.

### DESCRIPTION OF THE INVENTION

According to one aspect of the invention, there is provided a plurality of coated particles comprising:
(a) a solid core comprising a compound selected from the group consisting of lenalidomide and pomalidomide, or a pharmaceutically acceptable salt thereof, said core having a mean diameter of the between about 10 nm (such as about 0.1 μm) and about 700 μm (e.g. about 100 or about 50 μm); and
(b) a coating surrounding, enclosing and/or encapsulating said core, wherein the coating comprises a mixed oxide of at least two metal and/or metalloid oxides selected from the group consisting of iron oxide, zinc oxide, silicon oxide, titanium oxide, and aluminium oxide.

The examples provided hereinafter describe the production of the inventive coated lenalidomide particles and their advantageous in vitro release profile.

The term ‘solid’ will be well understood by those skilled in the art to include any form of matter that retains its shape and density when not confined, and/or in which molecules are generally compressed as tightly as the repulsive forces among them will allow. The solid cores have at least a solid exterior surface onto which a layer of coating material can be deposited. The interior of the solid cores may be also solid or may instead be hollow. For example, if the particles are spray dried before they are placed into the reactor vessel, they may be hollow due to the spray drying technique.

The solid cores may comprise lenalidomide and/or pomalidomide, or pharmaceutically-acceptable salt of either and, in this respect, may consist essentially of lenalidomide and/or pomalidomide or said salt thereof, or may include lenalidomide and/or pomalidomide or said salt thereof along with other excipients or other active ingredients.

By ‘consists essentially’ of lenalidomide and/or pomalidomide, or pharmaceutically-acceptable salt of either, we include that the solid cores are essentially comprised only of lenalidomide and/or pomalidomide or salt thereof, i.e. they are free from non-biologically active substances, such as excipients, carriers and the like (vide infra), and from other active substances. This means that cores may comprise less than about 5%, such as less than about 3%, including less than about 2%, e.g. less than about 1% of such other excipients and/or active substances.

In the alternative, cores comprising lenalidomide and/or pomalidomide or pharmaceutically-acceptable salt thereof may include that the active ingredient is in admixture with one or more pharmaceutical ingredients, which may include pharmaceutically-acceptable excipients, such as adjuvants, diluents or carriers, and/or may include other biologically-active ingredients.

Non-biologically active adjuvants, diluents and carriers that may be employed in cores to be coated in accordance with the invention may include pharmaceutically-acceptable substances that are soluble in water, such as carbohydrates, e.g. sugars, such as lactose and/or trehalose, and sugar alcohols, such as mannitol, sorbitol and xylitol; or pharmaceutically-acceptable inorganic salts, such as sodium chloride. Preferred carrier/excipient materials include sugars and sugar alcohols.

Lenalidomide and/or pomalidomide or pharmaceutically-acceptable salts of either may be presented in a crystalline, a part-crystalline and/or an amorphous state. Lenalidomide and/or pomalidomide or pharmaceutically-acceptable salt of either may be in the solid state, or may be converted into the solid state, at about room temperature (e.g. about 18° C.) and about atmospheric pressure, irrespective of the physical form. Active agent (and optionally other pharmaceutical ingredients as mentioned hereinbefore) should also remain in the form of a solid whilst being coated in, for example, an ALD reactor, and also should not decompose physically or chemically to an appreciable degree (i.e. no more than about 10% w/w) whilst being coated, or after having been covered by the mixed oxide coating material.

Pharmaceutically acceptable salts of lenalidomide and/or pomalidomide include acid addition salts and base addition salts. Such salts may be formed by conventional means, for example by reaction of a free acid or a free base form of a compound of the invention with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared using techniques known to those skilled in the art, such as by exchanging a counter-ion of a compound of the invention in the form of a salt with another counter-ion, for example using a suitable ion exchange resin.

Salts that may be mentioned include acid additional salts of, for example, hydrohalic acids (hydrochlorde acid), L-lactic acid, acetic acids (e.g. acetic acid and trifluoroacetic acid), phosphoric acid, (+)-L-tartaric acid, citric acid, propionic acid, butyric acid, hexanoic acid, L-aspartic acid, L-glutamic acid, succinic acid, ethylenediaminetetraacetic acid (EDTA), maleic acid, as well as alkyl- and arylsulfonic acids (including methanesulfonic acid, benzenesulfonic acid and toluenesulfonic acid), and the like. Particular salts that may be mentioned include lenalidomide benzenesulfonate, lenalidomide p-toluenesufonate and lenalidomide trihydrochloride, pomalidomide trifluoroacetate salts and pomalidomide salts.

The solid lenalidomide- and/or pomalidomide-containing cores of the invention are provided in the form of nanoparticles or, more preferably, microparticles. Preferred weight-, number-, or volume-based mean diameters are between about 50 nm (e.g. about 100 nm, such as about 250 nm) and about 30 μm, for example between about 500 nm and about 100 μm, more particularly between about 1 μm and about 50 μm, such as about 25 μm, e.g. about 20 μm.

According to one embodiment of this aspect of the invention, cores have a mean diameter of the between 3 μm and 20 μm, such as 5 μm to 15 μm, such as 8 μm to 12 μm, such as 9 μm to 11 μm, such as approximately 10 μm.

The term ‘mean diameter’ of the core as referred to herein, may be a weight-, a number-, or a volume-based mean diameter. As used herein, the term ‘weight based mean diameter’ will be understood by the skilled person to include that the average particle size is characterised and defined from a particle size distribution by weight, i.e. a distribution where the existing fraction (relative amount) in each size class is defined as the weight fraction, as obtained by e.g. sieving (e.g. wet sieving). As used herein, the term ‘number based mean diameter’ will be understood by the skilled person to include that the average particle size is characterised and defined from a particle size distribution by number, i.e. a distribution where the existing fraction (relative amount) in each size class is defined as the number fraction, as measured by e.g. microscopy. As used herein, the term ‘volume based mean diameter’ will be understood by the skilled person to include that the average particle size is characterised and defined from a particle size distribution by volume, i.e. a distribution where the existing fraction (relative amount) in each size class is defined as the volume fraction, as measured by e.g. laser diffraction. The person skilled in the art will also understand there are other suitable ways of expressing mean diameters, such as area based mean diameters, and that these other expressions of mean diameter are interchangeable with those used herein. Other instruments that are well known in the field may be employed to measure particle size, such as equipment sold by e.g. Malvern Instruments, Ltd (Worcestershire, UK) and Shimadzu (Kyoto, Japan).

Particles may be spherical, that is they possess an aspect ratio smaller than about 20, more preferably less than about 10, such as less than about 4, and especially less than about 2, and/or may possess a variation in radii (measured from the centre of gravity to the particle surface) in at least about 90% of the particles that is no more than about 50% of the average value, such as no more than about 30% of that value, for example no more than about 20% of that value.

Nevertheless, the coating of particles on any shape is also possible in accordance with the invention. For example, irregular shaped (e.g. ‘raisin’-shaped), needle-shaped, flake-shaped or cuboid-shaped particles may be coated. For a non-spherical particle, the size may be indicated as the size of a corresponding spherical particle of e.g. the same weight, volume or surface area. Hollow particles, as well as particles having pores, crevices etc., such as fibrous or ‘tangled’ particles may also be coated in accordance with the invention.

Particles may be obtained in a form in which they are suitable to be coated or be obtained in that form (and so at the size ranges specified herein), for example by particle size reduction processes (e.g. crushing, cutting, milling or grinding) to a specified weight based mean diameter (as hereinbefore defined), for example by wet grinding, dry grinding, air jet-milling (including cryogenic micronization), ball milling, such as planetary ball milling, as well as making use of end-runner mills, roller mills, vibration mills, hammer mills, roller mill, fluid energy mills, pin mills, etc. Alternatively, particles may be prepared directly to a suitable size and shape, for example by spray-drying, freeze-drying, spray-freeze-drying, vacuum-drying, precipitation, including the use of supercritical fluids or other top-down methods (i.e. reducing the size of large particles, by e.g. grinding, etc.), or bottom-up methods (i.e. increasing the size of small particles, by e.g. sol-gel techniques, crystallization, etc.). Nanoparticles may alternatively be made by well-known techniques, such as gas condensation, attrition, chemical precipitation, ion implantation, pyrolysis, hydrothermal synthesis, etc.

It may be necessary (depending upon how the particles that comprise the cores are initially provided) to wash and/or clean them to remove impurities that may derive from their production, and then dry them. Drying may be carried out by way of numerous techniques known to those skilled in the art, including evaporation, spray-drying, vacuum drying, freeze drying, fluidized bed drying, microwave drying, IR radiation, drum drying, etc. If dried, cores may then be deagglomerated by grinding, screening, milling and/or dry sonication. Alternatively, cores may be treated to remove any volatile materials that may be absorbed onto its surface, e.g. by exposing the particle to vacuum and/or elevated temperature.

Surfaces of cores may be chemically activated prior to applying the first layer of coating material, e.g. by treatment with hydrogen peroxide, ozone, free radical-containing reactants or by applying a plasma treatment, in order to create free oxygen radicals at the surface of the core. This in turn may produce favourable adsorption/nucleation sites on the cores for the ALD precursors.

As defined above, the mixed oxide refers to an oxide of more than one chemical element, in addition to oxygen. The term may refer to a mixture of more than one oxide of said chemical elements, including metals and/or metalloids as described herein. The mixed oxide may also be referred to as double oxide. The mixed oxide may be heterogenous or homogenous.

According to one embodiment of this aspect of the invention, the at least two metal and/or metalloid oxides in said mixed oxide are selected from the group consisting of iron oxide, more preferably zinc oxide, silicon oxide, titanium oxide, and aluminium oxide. According to the invention, at least one of the oxides may be zinc oxide or silicon oxide. It is preferred that at least one of the oxides is zinc oxide, such as the mixed oxide comprises mixtures of zinc oxide and aluminium oxide, or zinc oxide and silicon oxide.

In this respect, the lenalodimide- and/or polamlidomide-containing cores are preferably coated with a coating material that comprises a mixture of zinc oxide, and one or more other metal and/or metalloid oxides as described herein (e.g. titanium oxide, aluminium oxide or silicon oxide), at a atomic ratio of zinc oxide to the other oxide(s) that is at least about 1:6 (e.g. at least about 1:4, such as at least about 1:2), preferably at least about 1:1 (e.g. at least about 1.5:1, such as at least about 2:1), including at least about 2.25:1, such as at least about 2.5:1 (e.g. at least about 3.25:1 or least about 2.75:1 (including about 3:1)), and is up to (i.e. no more than) and including about 6:1, including up to about 5.5:1, or up to about 5:1, such as up to about 4.5:1, including up to about 4:1 (e.g. up to about 3.75:1).

Lenalidomide-containing cores may be coated with a coating material that comprises a mixed oxide of zinc oxide, and one or more other metal and/or metalloid oxides, at an atomic ratio of zinc oxide to the other oxide(s) that is at least about 2:1 (e.g. at least about 2.25:1, such as at least about 3.5:1, including at least about 2.5:1 (e.g. at least about 3.25:1 or least about 2.75:1 (including 3:1), and is no more that about 5:1, such as no more than about 4.5:1, including no more than about 4:1 (e.g. up to about 3.75:1). In one embodiment, said other oxide is aluminium oxide. In another embodiment, said other oxide is silicon oxide.

In the case of mixed oxides of the invention comprising mixtures of zinc oxide and silicon oxide, the atomic ratio of zinc to silicon in said mixed oxide may be between about 1 to 6 and up and including about 6 to 1, such as between about 1 to 4 and about 4 to 1, such as between 1 to 2.5 and about 3.5 to 1, such as 1 to 2, preferably between about 1 to 1 and up to and including about 6 to 1, such as between about 2 to 1 and about 5 to 1, such as between about 2 to 1 and about 4 to 1, such as between about 2.5 to 1 to about 3.5 to 1, such as about 3 to 1. In a further embodiment, the atomic ratio of zinc to silicon in said mixed oxide may be between about 1 to 1 and about 2 to 1, or between about 2 to 1 to about 3 to 1, or between about 3 to 1 and about 4 to 1.

In the case of mixed oxides of the invention comprising mixtures of zinc oxide and aluminium oxide, the atomic ratio of zinc to aluminium in said mixed oxide is between about 1 to 6 and up to and including about 6 to 1, such as between about 1 to 4 and about 4 to 1, such as between 1 to 2.5 and about 3.5 to 1, such as 1 to 2, preferably between about 1 to 1 and up and including about 6 to 1, such as between about 2 to 1 and about 5 to 1, such as between about 2 to 1 and about 4 to 1, such as between about 2.5 to 1 to about 3.5 to 1, such as about 3 to 1. In a further embodiment, the atomic ratio of zinc to aluminium in said mixed oxide is between about 1 to 1 and about 2 to 1, or between about 2 to 1 and about 3 to 1, or between about 3 to 1 and about 4 to 1.

Preferred methods of applying the coating(s) to the cores comprising biologically-active agents include gas phase techniques, such as ALD or related technologies, such as atomic layer epitaxy (ALE), molecular layer deposition (MLD; a similar technique to ALD with the difference that molecules (commonly organic molecules) are deposited in each pulse instead of atoms), molecular layer epitaxy (MLE), chemical vapor deposition (CVD), atomic layer CVD, molecular layer CVD, physical vapor deposition (PVD), sputtering PVD, reactive sputtering PVD, evaporation PVD and binary reaction sequence chemistry. ALD is the preferred method of coating according to the invention.

According to one embodiment of this aspect of the invention, the total thickness of the coating(s) applied to the cores is between about 0.2 nm and about 2 μm. In one embodiment, said total thickness of the coating is between about 0.5 nm and about 2 μm, such as between about 0.5 nm and about 2 μm, such as between about 1 nm and about 2 μm, such as between about 2 nm and about 2 μm. In one embodiment, said total thickness of the coating is between about 0.5 nm and about 500 nm, such as between about 1 nm and about 400 nm, such as between about 2 nm and about 300 nm. In one embodiment, said total thickness of the coating is between about 0.5 nm and about 50 nm, such as between about 1 nm and about 40 nm, such as between about 1 nm and about 30 nm, such as such as between about 1 nm and about 25 nm, such as between about 1 nm and about 20 nm, such as between about 1 nm and about 15. In one embodiment, said total thickness of the coating is between about 2 nm and about 30 nm, such as between about 3 nm and about 30 nm, such as between about 4 nm and about 30 nm or between about 3 nm and about 20 nm, such as between about 4 nm and about 20 nm. In one embodiment, said total thickness of the coating is between about 2 nm and about 15 nm, such as between about 3 nm and about 15 nm, such as between about 3 nm and about 10 nm, such as between about 4 nm and about 15 nm, such as between about 4 nm and about 10 nm. In one embodiment, said total thickness of the coating is between about 5 nm and about 10 nm.

The minimum thickness of each individual layer will on average be in the region of about 0.1, more particularly about 0.3 nm, including about 0.5 nm, for example about 0.75 nm, such as about 1 nm (including about 1.5 nm, for example about 2.25 nm, such as about 3 nm, such as about 5 nm, such as about 10 nm).

The average thickness of each individual layer may be at least 0.3 nm, such as about 1.5 nm, such about 2.25 nm, such as about 3 nm, such as about 5 nm, such as about nm.

The maximum thickness of each individual layer/coating/shell will depend on the size of the core (to begin with), and thereafter the size of the core with the coatings that have previously been applied, and may be on average about 1 hundredth of the mean diameter (i.e. the weight-, number-, or volume-based mean diameter) of that core, or core with previously applied coatings.

Preferably, for particles with a mean diameter that is between about 100 nm and about 1 μm, the total coating thickness should be on average between about 1 nm and about nm; for particles with a mean diameter that is between about 1 μm and about 20 μm, the coating thickness should be on average between about 1 nm and about 10 nm; for particles with a mean diameter that is between about 20 μm and about 700 μm, the coating thickness should be on average between about 1 nm and about 100 nm.

When ALD is employed, the above-described mixed oxide coating may be prepared by feeding a first, zinc-, or other metal- or metalloid-containing precursor, into an ALD reactor chamber (in a so called ‘precursor pulse’) to form the adsorbed atomic or molecular (e.g. zinc-, other metal- or metalloid-containing) monolayer at the surface of the particle. A second precursor (e.g. water) is then pulsed into the reactor and reacts with the first precursor, resulting in the formation of a monolayer of zinc, metal or metalloid oxide, respectively, on the substrate surface. A subsequent purging pulse is followed by a further pulse of the first precursor, and thus the start of a new cycle of the same events, which is an ALD cycle.

In most instances, the first of the consecutive reactions will involve some functional group or free electron pairs or radicals at the surface to be coated, such as a hydroxy group (—OH) or a primary or secondary amino group (—NH₂ or —NHR where R e.g. is an aliphatic group, such as an alkyl group). The individual reactions are advantageously carried out separately and under conditions such that all excess reagents and reaction products are essentially removed before conducting the subsequent reaction.

According to a further aspect of the invention, there is provided a plurality of particles, which particles:
(a) have a weight-, number-, or volume-based mean diameter that is between 10 nm (such as about 0.1 μm) and about 700 μm (e.g. about 100 or about 50 μm, or one or more of the particle size ranges described herein); and
(b) comprise solid cores comprising a compound selected from the group consisting of lenalidomide and pomalidomide, or a pharmaceutically-acceptable salt of either compound, coated, at least in part, by a coating of inorganic material comprising mixture of:
   (i) zinc oxide (ZnO); and
   (ii) one or more other metal and/or metalloid oxides, wherein the atomic ratio ((i):(ii)) is at least about 1:6 and up to and including about 6:1.

Preferably, there is provided a plurality of particles, in which particles the atomic ratio ((i):(ii)) is at least about 1:1 and up to and including about 6:1.

In order to make a mixed oxide coating with a atomic ratio of (for example) between about 1:1 and about 6:1 of a metal oxide, such as zinc oxide, relative to the one or more other metal and/or metalloid oxide, the skilled person will appreciate that for every one ALD cycle (i.e. monolayer) of the other oxide(s), between about 1 and about 6 ALD cycles of e.g. zinc oxide must also be deposited. For example, for a 3:1 weight ratio (e.g. zinc:other oxide) mixed oxide coating to be formed, 3 e.g. zinc-containing precursor pulses may each be followed by second precursor pulses, forming 3 monolayers of e.g. zinc oxide, which will then be followed by 1 pulse of the other metal and/or metalloid-containing precursor followed by second precursor pulse, forming 1 monolayer of an oxide of the other metal and/or metalloid. Alternatively, 6 monolayers of e.g. zinc oxide may be followed by 2 monolayers of the other oxide, or any other combination so as to provide an overall atomic ratio of about 3:1. In this respect, the order of pulses to produce the relevant oxides is not critical, provided that the resultant atomic ratio is in the relevant range in the end.

Metal and/or metalloid elements other than zinc that may be mentioned include alkali metals, alkaline earth metals, noble metals, transition metals, post-transition metals, lanthanoids, etc. Metal and/or metalloids that may be mentioned include aluminium, titanium, magnesium, iron, gallium, zirconium, niobium, hafnium, tantalum, lanthanum, and/or silicon; more preferably aluminium, titanium, magnesium, iron, gallium, and/or zirconium. Particular metal and/or metalloid elements that may be mentioned include aluminium and silicon.

The mixed oxide coating material thus preferably comprises one or other or both of aluminium oxide (Al₂O₃) and/or silicon dioxide (SiO₂).

In ALD, layers of coating materials may be applied at process temperatures from about 20° C. to about 800° C., or from about 40° C. to about 200° C., e.g. from about 40° C. to about 150° C., such as from about 50° C. to about 100° C. The optimal process temperature depends on the reactivity of the precursors and/or substances (including biologically-active agents, such as lenalidomide or pomalidomide or salts of either) that are employed in the core and/or melting point of the core substance(s). It is preferred that a lower temperature, such as from about 30° C. to about 100° C. is employed. In particular, in one embodiment of the method a temperature from about 20° C. to about 80° C. is employed, such as from about 30° C. to about 70° C., such as from about 40° C. to about 60° C., such as about 50° C.

We have found that, when coatings comprising zinc oxide are applied using ALD at a lower temperature, such as from about 50° C. to about 100° C. (unlike other coating materials, such as aluminium oxide and titanium oxide, which form amorphous layers) the coating materials are largely crystalline in their nature.

Without being limited by theory, because zinc oxide is crystalline, if only zinc oxide is employed as coating material, we are of the understanding that interfaces may be formed between adjacent crystals of zinc oxide that are deposited by ALD, through which a carrier system, medium or solvent in which zinc oxide is partially soluble (e.g. an aqueous solvent system) can ingress following suspension therein. It is believed that this may give rise to dissolution that is too fast for the depot-forming composition that it is intended to make.

In addition, previous studies have shown that, when suspended in aqueous media, the relative bioavailability for formulations comprising an active ingredient that has been coated with zinc oxide is lower than uncoated active ingredient. We believe that this lower relative bioavailability is due to degradation of the active before it can be released into systemic circulation. Penetration of water through crystalline interfaces within a zinc oxide coating as described above is thought to lead to hydrolysis of the active ingredient within the interior of the coated particle.

We have now found that these problems may be alleviated by making a mixed oxide coating as described herein. In particular, we have now found that these problems may be alleviated by making a mixture of two or more metal and/or metalloid oxides (mixed oxide) coating as defined herein. In particular, by forming a mixed oxide coating as described herein, that may be predominantly, but not entirely, comprised of zinc oxide, we have been able to coat active ingredients with coatings that appear to be essentially amorphous, or a composite between crystalline and amorphous material, and/or in which ingress of injection vehicles such as water may be reduced. In this respect, it appears to us that the presence of the aforementioned perceived interfaces may be reduced, or avoided altogether, by employing the mixed oxide aspect of the invention, in either a heterogeneous manner (in which the other oxide is ‘filling in’ gaps formed by the interfaces), or in a homogeneous manner (in which a true composite of mixed oxide materials is formed during deposition, in a manner where the interfaces are potentially avoided in the first place). As described hereinafter, formulations according to the invention demonstrate relative bioavailabilities that are comparable with the uncoated lenalidomide or pomalidomide.

According to the invention, there is provided a method of preparing of plurality of coated particles according to the invention, wherein the coated particles are made by applying precursors of at least two metal and/or metalloid oxides forming a mixed oxide on the solid cores, and/or previously-coated solid cores, by a gas phase deposition technique. Precursors for forming a metal oxide or a metalloid oxide often include an oxygen precursor, such as water, oxygen, ozone and/or hydrogen peroxide; and a metal and/or metalloid compound, typically an organometal compound or an organometalloid compound.

Non-limiting examples of precursors are as follows: Precursors for zinc oxide may be water and diC₁-C₅alkylzinc, such as diethylzinc. Precursors for aluminium oxide may be water and triC₁-C₅alkylaluminium, such as trimethylaluminium. Precursors for silicon oxide (silica) may be water as the oxygen precursor and silanes, alkylsilanes, aminosilanes, and orthosilicic acid tetraethyl ester. Precursors for iron oxide includes oxygen, ozone and water as the oxygen precursor; and di C₁-C₅alkyl-iron, dicyclopropyl-iron, and FeCl₃. It will be appreciated that the person skilled in the art is aware of what precursors are suitable for the purpose as disclosed herein.

The gas phase deposition reactor chamber used may optionally, and/or preferably, be a stationary gas phase deposition reactor chamber. The term ‘stationary’, in the context of gas phase deposition reactor chambers, will be understood to mean that the reactor chamber remains stationary while in use to perform a gas phase deposition technique, excluding negligible movements and/or vibrations such as those caused by associated machinery for example.

Additionally, a so-called ‘stop-flow’ process may be employed. Using a stop-flow process, once the first precursor has been fed into the reactor chamber and prior to the first precursor being purged from the reactor chamber, the first precursor may be allowed to contact the cores in the reactor chamber for a pre-determined period of time (which may considered as a soaking time). During the pre-determined period of time there is preferably a substantial absence of pumping that may result in flow of gases and/or a substantial absence of mechanical agitation of the cores.

The employment of the stop-flow process may increase coating uniformity by allowing each gas to diffuse conformally in high aspect-ratio substrates, such as powders. The benefits may be even more pronounced when using precursors with slow reactivity as more time is given for the precursor to react on the surface. This may be evident especially when depositing mixed oxide coatings according to the invention. For example, when depositing a mixed zinc oxide/aluminium oxide coating as described herein, we have found that a zinc-containing precursor, such as diethylzinc (DEZ), which has a lower reaction probability towards the surface of a substrate than, for example, aluminium containing precursors, such as trimethylaluminum (TMA).

In addition to generating coatings with good shell integrity and more controlled release profiles, the employment of such a stop-flow process may improve the ability to achieve a particular coating composition.

For example, when attempting to employ a gas phase technique to produce a coating comprising an atomic ratio of 3:1 between zinc and aluminium in the resulting shell as described above, we have found that a ratio that is much closed to 3:1 may be achieved using a stop-flow process than when depositing material using a continuous flow of precursors.

Preferably, and/or optionally, a ‘multi-pulse’ technique may also be employed to feed the first precursor, the second precursor or both precursors to the reactor chamber. Using such a multi-pulse technique, the respective precursor may be fed into the reactor chamber as a plurality of ‘sub-pulses’, each lasting a short period of time such as 1 second up to about a minute (depending on the size and the nature of the gas phase deposition reactor), rather than as one continuous pulse. The precursor may be allowed to contact the cores in the reactor chamber for the pre-determined period of time, for example from about 1 to 500 seconds, about 2 to 250 seconds, about 3 to 100 seconds, about 4 to 50 seconds, or about 5 to 10 seconds, for example 9 seconds, after each sub-pulse. Again, depending on the size and the nature of the gas phase deposition reactor, this time could be extended up to several minutes (e.g. up to about 30 minutes). The introduction of a sub-pulse followed by a period of soaking time may be repeated a pre-determined number of times, such as between about 5 to 1000 times, about 10 to 250 times, or about 20 to 50 times in a single step.

The process according to the invention may further comprise the steps of:
(1) applying precursors of one or more metal and/or metalloid oxides selected from the group consisting of iron oxide, zinc oxide, silicon oxide, titanium oxide, and aluminium oxide, whereby a layer is formed on said solid cores by way of a gas phase deposition technique;
(2) disaggregating the coated solid core aggregates formed during step (1);
(3) applying precursors of at least one metal and/or metalloid oxides selected from the group consisting of iron oxide, zinc oxide, silicon oxide, titanium oxide, and aluminium oxide to the disaggregated, coated solid cores from step (2) whereby another layer is formed on said previously-coated solid cores by way of a gas phase deposition technique; and
(4) repeating steps (2) and (3) one or more times to increase the total thickness of the coating that encloses said solid core,
wherein at least one of the layers is formed by applying precursors of at least two metal and/or metalloid oxides selected from the group consisting of iron oxide, zinc oxide, silicon oxide, titanium oxide, and aluminium oxide, whereby a mixed oxide is formed. Thus, at least one of said layers in steps 1, 3 or 4 comprises a mixed oxide.

In this respect, the cores may be coated in accordance with the invention with one or more separate, discrete layers, of mixed oxide coatings as defined herein. Preferably, more than one separate, discrete mixed oxide layer, coating or shell (which terms are used herein interchangeably) is applied (that is ‘separately applied’) to the solid cores comprising active ingredient sequentially.

By ‘separate application’ of ‘separate layers, coatings or shells’, we mean that the solid cores are coated with a first layer of coating material, which layer is formed by more than one (e.g. a plurality or a set of) cycles as described herein, each cycle producing a monolayer of zinc oxide, or other metal and/or metalloid oxide (as appropriate), and then that resultant coated core is subjected to some form of deagglomeration process.

In other words, ‘gas-phase deposition (e.g. ALD) cycles’ can be repeated several times to provide a ‘gas-phase deposition (e.g. ALD) set’ of cycles, which may consist of e.g. 10, 25 or 100 cycles. However, after this set of cycles, the coated core is subjected to some form of deagglomeration process, which is followed by a further set of cycles.

This process may be repeated as many times as is desired and, accordingly, the number of discrete layers of coating material(s) produced by sets of cycles that is in a final coating corresponds to the number of these intermittent deagglomeration steps with a final mechanical deagglomeration being conducted prior to the application of a final layer (set of cycles) of coating material.

According to the invention, the coating comprises 1 to about 20 of such discrete layers, such as 1 to about 10 discrete layers, 2 to 8 discrete layers, wherein one or more mixed oxides. Said coatings may comprises such as 1 to 19, such as 1 to 18, such as 1 to 17, such as 1 to 16, such as 1 to 15, such as 1 to 14, such as 1 to 13, such as 1 to 12, such as 1 to 11, such as 1 to 10, such as 1 to 9, such as 1 to 8, such as 1 to 7, such as 1 to 6 of said discrete layers. In another embodiment, said coating comprises 2 to 20, such as 2 to 19, such as 2 to 18, such as 2 to 17, such as 2 to 16, such as 2 to 15, such as 2 to 14, such as 2 to 13, such as 2 to 12, such as 2 to 11, such as 2 to 10, such as 2 to 9, such as 2 to 8, such as 2 to 7, such as 2 to 6 of said discrete layers. In another embodiment said coating comprises 3 to 20, such as 3 to 19, such as 3 to 18, such as 3 to 17, such as 3 to 16, such as 3 to 15, such as 3 to 14, such as 3 to 13, such as 3 to 12, such as 3 to 11, such as 3 to 10, such as 3 to 9, such as 3 to 8, such as 3 to 7, such as 3 to 6 of said discrete layers. In a further embodiment, said coating comprises 4 to 20, such as 4 to 19, such as 4 to 18, such as 4 to 17, such as 4 to 16, such as 4 to 15, such as 4 to 14, such as 4 to 13, such as 4 to 12, such as 4 to 11, such as 4 to 10, such as 4 to 9, such as 4 to 8, such as 4 to 7, such as 4 to 6 of said discrete layers. In particular embodiments, said coating may comprises 2, 3, 4, 5, 6, 7, 8, 9 or 10 of said discrete layers, including between 3 and 7, such as between 4 and 6 of said discrete layers.

The separate, discrete layers formed in steps 1 and/or 3 (and 4) of the process described above may comprise depositions from one or several ALD cycles as described and/or defined herein. Typically, each separate, discrete layer comprises the deposition from 1 to 50 ALD cycles, such as 10 to 40 ALD cycles, such as 15 to 35 ALD cycles, such as 20 to 30 ALD cycles, such as 22 to 26 ALD cycles. According to the invention, the terms ‘disaggregation’ and ‘deagglomeration’ are used interchangeably when referring to the coated particles, and disaggregating coated particles aggregates is preferably done by way of a mechanical sieving technique.

We have found that applying coatings/shells followed by conducting one or more deagglomeration step such as sonication gives rise to abrasions, pinholes, breaks, gaps, cracks and/or voids (hereinafter ‘cracks’) in the layers/coatings, due to coated particles essentially being more tightly ‘bonded’ or ‘glued’ together directly after the application of a thicker coating. This may expose a core comprising biologically-active ingredient to the elements once deagglomeration takes place.

As it is intended to provide particles in an aqueous suspension prior to administration to a patient, it is necessary to provide deagglomerated primary particles without pinholes or cracks in the coatings. Such cracks will result in an undesirable initial peak (burst) in plasma concentration of active ingredient directly after administration.

We have found that, by conducting one or more of the deagglomeration steps more specifically described hereinafter, this gives rise to significantly less pinholes, gaps or cracks in the final layer of coating material, giving rise to particles that are not only completely covered by that layer/coating, but are also covered in a manner that enables the particles to be deagglomerated readily (e.g. using a non-aggressive technique, such as vortexing) in a manner that does not destroy the layers of coating material that have been formed, prior to, and/or during, pharmaceutical formulation.

In this respect, the mixed oxide coating typically completely surrounds, encloses and/or encapsulates said solid cores comprising active ingredient(s). In this way, the risk of an initial drug concentration burst due to the drug coming into direct contact with solvents in which the relevant active ingredient is soluble is minimized. This may include not only bodily fluids, but also any medium in which such coated particles may be suspended prior to injection.

Thus, in one embodiment of the invention, there are provided particles as hereinbefore disclosed, wherein said coating surrounding, enclosing and/or encapsulating said core covers at least about 50%, such as at least about 65%, including at least about 75%, such as at least about 80%, more particularly at least about 90%, such as at least about 91%, such as at least about 92%, such as at least about 93%, such as at least about 94%, such as at least about 95%, such as at least about 96%, such as at least about 97%, such as at least about 98%, such as at least about 99%, such as approximately, or about, 100%, of the surface of the solid core, such that the coating essentially completely surrounds, encloses and/or encapsulates said core.

As used herein, the term ‘essentially completely coating completely surrounds, encloses and/or encapsulates said core’ means a covering of at least about 98%, or at least about 99%, of the surface of the solid core.

In the alternative, processes described herein may result in the deagglomerated coated particles with the essential absence of said cracks through which active ingredient can be released in an uncontrolled way.

Although some minor cracks may appear in the said coating without effecting the essential function thereof in terms of controlling release, in a further embodiment, there are provided particles as hereinbefore disclosed, wherein at least about 90% of the particles do not exhibit cracks in the coating surrounding, enclosing and/or encapsulating said core. In one embodiment at least about 91%, such as at least about 92%, such as at least about 93%, such as at least about 94%, such as at least about 95%, such as at least about 96%, such as at least about 97%, such as at least about 98%, such as at least about 99%, such as approximately 100% of the particles do not exhibit said cracks.

Alternatively, by ‘essentially free of said cracks’ in the coating(s), we mean that less than about 1% of the surfaces of the coated particles comprise abrasions, pinholes, breaks, gaps, cracks and/or voids through which active ingredient is potentially exposed (to, for example, the elements).

Coated cores may be subjected to the aforementioned deagglomeration process internally, without being removed from said apparatus by way of a continuous process. Such a process will involve forcing solid product mass formed by coating said cores through a sieve that is located within the reactor, and is configured to deagglomerate any particle aggregates upon forcing of the coated cores by means of a forcing means applied within said reactor, prior to being subjected to a second and/or a further coating. This process is continued for as many times as is required and/or appropriate prior to the application of the final coating as described herein.

Having the sieve located within the reactor vessel means that the coating can be applied by way of a continuous process which does not require the particles to be removed from the reactor. Thus, no manual handling of the particles is required, and no external machinery is required to deagglomerate the aggregated particles. This not only considerably reduces the time of the coating process being carried out but is also more convenient and reduces the risk of harmful (e.g. poisonous) materials being handled by personnel. It also enhances the reproducibility of the process by limiting the manual labour and reduces the risk of contamination.

According to one embodiment of this aspect of the invention, step 2 further comprises a step of discharging the coated solid cores from the gas phase deposition reactor, prior to disaggregating coated solid core aggregates formed during step (1); and the step of charging the coated solid cores back to the reactor subsequent to disaggregating coated solid cores aggregates formed during step (1).

According to one embodiment of this aspect of the invention, the gas phase deposition technique is selected from atomic layer deposition, chemical vapour deposition, and physical vapour deposition.

According to one embodiment of this aspect of the invention, the gas phase deposition technique is atomic layer deposition (ALD). It will be appreciated that ALD may be particularly useful for the deposition of layers on complex and uneven structures having areas or surfaces which are difficult to access.

According to one embodiment of this aspect of the invention, said precursors are:
an oxygen containing precursor, such as water, oxygen, ozone and/or hydrogen peroxide; and
a metal and/or metalloid compound selected from diethylzinc, trimethylaluminium, and orthosilicic acid tetraethyl ester.

Alternatively, and/or preferably, coated cores may be removed from the coating apparatus, such as the ALD reactor, and thereafter subjected to an external deagglomeration step, for example as described in international patent application WO 2014/187995. Such an external deagglomeration step may comprise agitation, such as sonication in the wet or dry state, or preferably may comprise subjecting the resultant solid product mass that has been discharged from the reactor to sieving, e.g. by forcing it through a sieve or mesh in order to deagglomerate the particles, for example as described hereinafter, prior to placing the particles back into the coating apparatus for the next coating step. Again, this process may be continued for as many times as is required and/or appropriate prior to the application of the final coating.

In an external deagglomeration process, deagglomeration may alternatively be effected (additionally and/or instead of the abovementioned processes) by way of subjecting the coated particles in the wet or dry state to one or more of nozzle aerosol generation, milling, grinding, stirring, high sheer mixing and/or homogenization. If the step(s) of deagglomeration are carried out on particles in the wet state, the deagglomerated particles should be dried (as hereinbefore described in relation to cores) prior to the next coating step.

However, we prefer that, in such an external process, the deagglomeration step(s) comprise one or more sieving step(s), which may comprise jet sieving, manual sieving, vibratory sieve shaking, horizontal sieve shaking, tap sieving, or (preferably) sonic sifting as described hereinafter, or a like process, including any combination of these sieving steps. The skilled person is aware of different appropriate sieving techniques that may be used for the purpose disclosed herein. Manufacturers of suitable sonic sifters include Advantech Manufacturing, Endecott and Tsutsui.

Vibrational sieving techniques may involve a means of vibrationally forcing the solid product mass formed by coating said cores through a sieve that is located internally or (preferably) externally to (i.e. outside of) the reactor, and is configured to deagglomerate any particle aggregates upon said vibrational forcing of the coated cores, prior to being subjected to a second and/or a further layer of coating material. This process is repeated as many times as is required and/or appropriate prior to the application of a final layer of coating material.

Vibrational forcing means comprises a vibration motor which is coupled to a sieve. The vibration motor is configured to vibrate and/or gyrate when an electrical power is supplied to it. For example, the vibration motor may be a piezoelectric vibration motor comprising a piezoelectric material which changes shape when an electric field is applied, as a consequence of the converse piezoelectric effect. The changes in shape of the piezoelectric material cause acoustic or ultrasonic vibrations of the piezoelectric vibration motor.

The vibration motor may alternatively be an eccentric rotating mass (ERM) vibration motor comprising a mass which is rotated when electrical power is supplied to the motor. The mass is eccentric from the axis of rotation, causing the motor to be unbalanced and vibrate and/or gyrate due to the rotation of the mass. Further, the ERM vibration motor may comprise a plurality of masses positioned at different locations relative to the motor. For example, the ERM vibration motor may comprise a top mass and a bottom mass each positioned at opposite ends of the motor. By varying each mass and its angle relative to the other mass, the vibrations and/or gyrations of the ERM vibration motor can be varied.

The vibration motor is coupled to the sieve in a manner in which vibrations and/or gyrations of the motor when electrical power is supplied to it are transferred to the sieve.

The sieve and the vibration motor may be suspended from a mount (such as a frame positionable on a floor, for example) via a suspension means such that the sieve and motor are free to vibrate relative to the mount without the vibrations being substantially transferred to or dampened by the mount. This allows the vibration motor and sieve to vibrate and/or gyrate without impediment and also reduces noise generated during the vibrational sieving process. The suspension means may comprise one or more springs or bellows (i.e. air cushion or equivalent cushioning means) that couple the sieve and/or motor to the mount. Manufacturers of vibratory sieves or sifters suitable for carrying out such a process include for instance Russell Finex, SWECO, Filtra Vibracion, VibraScreener, Gough Engineering and Farley Greene.

Preferably, the vibrational sieving technique further comprises controlling a vibration probe coupled to the sieve. The vibration probe may be controlled to cause the sieve to vibrate at a separate frequency to the frequency of vibrations caused by the vibration motor. Preferably the vibration probe causes the sieve to vibrate at a higher frequency than the vibrations caused by the vibration motor and, more preferably, the frequency is within the ultrasonic range.

Providing additional vibrations to the sieve by means of the vibration probe reduces the occurrence of clogging in the sieve, reduces the likelihood of the sieve being overloaded and decreases the amount of time needed to clean the mesh of the sieve.

Preferably, the aforesaid vibrational sieving technique comprises sieving coated particles with a throughput of at least 1 g/minute. More preferably, the vibrational sieving technique comprises sieving coated particles with a throughput of 4 g/minute or more.

The throughput depends on the area of the sieve mesh, mesh-size of the sieve, the particle size, the stickiness of the particles, static nature of the particle. By combining some of these features a much higher throughput is possible. Accordingly, the vibrational sieving technique may more preferably comprise sieving coated particles with a throughput of up to 1 kg/minute or even higher.

Any one of the above-stated throughputs represents a significant improvement over the use of known mechanical sieving, or sifting, techniques. For example, we found that sonic sifting involved sifting in periods of 15 minutes with a 15-minute cooling time in-between, which is necessary for preserving the apparatus. To sift 20 g of coated particles required 9 sets of 15 minutes of active sifting time, i.e. a total time (including the cooling) of 255 minutes. By comparison, by using the aforementioned vibrational sieving technique, 20 g of coated particles may be sieved continuously in, at most, 20 minutes, or more preferably in just 5 minutes, or less.

The sieve mesh size may be determined so that the ratio of the size of the sieved or sonic sifted particles to the sieve mesh size is about 1:>1, preferably about 1:2, and optionally about 1:4. The size mesh size may range from about 20 μm to about 100 μm, preferably from about 20 μm to about 60 μm.

Appropriate sieve meshes may include perforated plates, microplates, grid, diamond, threads, polymers or wires (woven wire sieves) but are preferably formed from metals, such as stainless steel.

Surprisingly, using a stainless steel mesh within the vibrational sieving technique is as gentle to the particle coatings as using a softer polymer sieve as part of a mechanical sieving technique such as sonic sifting.

Also, a known problem with sieving powders is the potentially dangerous generation of static electricity. A steel mesh has the advantage of removing static electricity from the powder while that is not the case with a polymeric mesh, which has to be used in a sonic sifter.

Further, the mesh size of known sonic sifters is limited to about 100 μm since the soundwaves travel through the mesh rather than vibrating it. That limitation does not exist using for vibrational sieving techniques as there is no reliance on soundwaves to generate vibrations in the sieve. Therefore, the vibrational sieving technique described herein allows larger particles to be sieved than if alternative mechanical sieving techniques were used.

If a (e.g. vibrational) sieve is located externally to (i.e. outside of) the reactor, the process for making coated cores of formulations of the invention comprises discharging the coated particles from the gas phase deposition reactor prior to subjecting the coated particles to agitation, followed by reintroducing the deagglomerated, coated particles into the gas phase deposition reactor prior to applying a further layer of at least one coating material to the reintroduced particles.

We have found that applying separate layers of coating materials following external deagglomeration gives rise to visible and discernible interfaces that may be observed by analysing coated particles according to the invention, and are observed by e.g. TEM as regions of higher electron permeability. In this respect, the thickness of the layers between interfaces correspond directly to the number of cycles in each series that are carried out within the ALD reactor, and between individual external agitation steps.

Because, in an ALD coating process, coating takes place at the atomic level, such clear, physical interfaces are typically more difficult to observe.

Without being limited by theory, it is believed that removing coated particles from the vacuum conditions of the ALD reactor and exposing a newly-coated surface to the atmosphere results in structural rearrangements due to relaxation and reconstruction of the outermost atomic layers. Such a process is believed to involve rearrangement of surface (and near surface) atoms, driven by a thermodynamic tendency to reduce surface free energy.

Furthermore, surface adsorption of species, e.g. hydrocarbons that are always present in the air, may contribute to this phenomenon, as can surface modifications, due to reaction of coatings formed with hydrocarbons, as well as atmospheric oxygen and the like. Accordingly, if such interfaces are analysed chemically, they may contain traces of contaminants or the core material, such as active ingredient that forms part of the core, that do not originate from the coating process, such as ALD.

Whether carried out inside or outside of the reactor, particle aggregates are preferably broken up by a forcing means that forces them through a sieve, thus separating the aggregates into individual particles or aggregates of a desired and predetermined size (and thereby achieving deagglomeration). In the latter regard, in some cases the individual primary particle size is so small (i.e. <1 μm) that achieving ‘full’ deagglomeration (i.e. where aggregates are broken down into individual particles) is not possible. Instead, deagglomeration is achieved by breaking down larger aggregates into smaller aggregates of secondary particles of a desired size, as dictated by the size of the sieve mesh. The smaller aggregates are then coated by the gas phase technique to form fully coated ‘particles’ in the form of small aggregate particles. In this way, the term ‘particles’, when referring to the particles that have been deagglomerated and coated in the context of the invention, refers to both individual (primary) particles and aggregate (secondary) particles of a desired size.

In any event, the desired particle size (whether that be of individual particles or aggregates of a desired size) is maintained and, moreover, continued application of the gas phase coating mechanism to the particles after such deagglomeration via the sieving means that a complete coating is formed on the particle, thus forming fully coated particles (individual or aggregates of a desired size).

Whether carried out inside or outside of the reactor, the above-described repeated coating and deagglomeration process may be carried out at least 1, preferably 2, more preferably 3, such as 4, including 5, more particularly 6, e.g. 7 times, and no more than about 100 times, for example no more than about 50 times, such as no more than about 40 times, including no more than about 30 times, such as between 2 and times, e.g. between 3 and 15 times, such as 10 times, e.g. 9 or 8 times, more preferably 6 or 7 times, and particularly 4 or 5 times.

According to this aspect of the invention, the mechanical sieving technique is sonic sifting and/or is vibrational sieving.

The layers of coating material may, taken together, be of an essentially uniform thickness over the surface area of the particles. By ‘essentially uniform thickness, we mean that the degree of variation in the thickness of the coating of at least about 10%, such as about 25%, e.g. about 50%, of the coated particles that are present in a composition of the invention, as measured by TEM, is no more than about ±20%, such as no more than about ±50% of the average thickness.

According to one embodiment of this aspect of the invention, the coating comprises 1 to 10 layers, such as 3 to 7 or 4 to 6 layers, all comprising a mixture, i.e. a mixed oxide, of zinc oxide and aluminium oxide, wherein each layer is applied using 20 to 30 ALD cycles, such as 22 to 26 ALD cycles, wherein the atomic ratio of zinc oxide to aluminium oxide is between 2 to 1 and 4 to 1.

According to one embodiment of this aspect of the invention, the total number of ALD cycles used for obtaining the coating is approximately 80 to approximately 180, such as approximately 90 to approximately 150 cycles. In particular embodiments, the total number of ALD cycles used for obtaining the coating approximately 100 to approximately 400, such as approximately 200 to approximately 300 cycles.

In addition to the essential mixed oxide coating that is employed in formulations of the invention, other coating materials, which may be pharmaceutically-acceptable and essentially non-toxic coating materials may also be applied in addition, either between separate mixed oxide coatings (e.g. in-between separate deagglomeration steps) and/or whilst a mixed oxide coating is being applied herein. Such materials may comprise multiple layers or composites of the mixture of metal and/or metalloid oxides (mixed oxide) and one or more different inorganic or organic materials, to modify the properties of the layer(s).

Additional coating materials may comprise organic or polymeric materials, such as a polyamide, a polyimide, a polyurea, a polyurethane, a polythiourea, a polyester or a polyimine. Additional coating materials may also comprise hybrid materials (as between organic and inorganic materials), including materials that are a combination between a metal, or another element, and an alcohol, a carboxylic acid, an amine or a nitrile. However, we prefer that coating materials comprise inorganic materials.

Additional inorganic coating materials may comprise other compounds of metals and/or or metalloids, such as oxides, nitrides, sulphides, selenides, carbonates, other ternary compounds, etc. Metal, and metalloid, hydroxides and, especially, oxides are preferred, especially metal oxides.

In addition, oxides of elements other than zinc, aluminium or silicon that may be mentioned include alkali metals, alkaline earth metals, noble metals, transition metals, post-transition metals, lanthanoids. Metal and metalloids that may be mentioned include titanium, magnesium, iron, gallium, zirconium, niobium, hafnium, tantalum and/or lanthanum; more preferably titanium, magnesium, iron, gallium and/or zirconium.

Additional coating materials that may be mentioned, thus, include those comprising titanium dioxide (TiO₂), iron oxides (FexOy, e.g. FeO and/or Fe₂O₃ and/or Fe₃O₄), gallium oxide (Ga₂O₃), magnesium oxide (MgO), niobium oxide (Nb₂O₅), hafnium oxide (HfO₂), tantalum oxide (Ta₂O₅), lanthanum oxide (La₂O₃) and/or zirconium dioxide (ZrO₂).

Although the plurality of mixed oxide coated particles in accordance with the invention are essentially free of the aforementioned cracks in the applied coatings, through which active ingredient is potentially exposed (to, for example, the elements), two further, optional steps may be applied to the plurality of coated particles prior to subjecting it to further pharmaceutical formulation processing.

The first optional step may comprise, subsequent to the final deagglomeration step as hereinbefore described, application of a final overcoating layer, the thickness of which outer ‘overcoating’ layer/coating, or ‘sealing shell’ (which terms are used herein interchangeably), must be thinner than the previously-applied separate layers/coatings/shells (or ‘subshells’).

The thickness may therefore be on average no more than a factor of about 0.7 (e.g. about 0.6) of the thickness of the widest previously-applied subshell. Alternatively, the thickness may be on average no more than a factor of about 0.7 (e.g. about 0.6) of the thickness of the last subshell that is applied, and/or may be on average no more than a factor of about 0.7 (e.g. about 0.6) of the average thickness of all of the previously-applied subshells. The thickness may be on average in the region of about 0.3 nm to about 10 nm, for particles up to about 20 μm. For larger particles, the thickness may be on average no more than about 1/1000 of the coated particles' weight-, number-, or volume-based mean diameter.

The role of such as sealing shell is to provide a ‘sealing’ overcoating layer on the particles, covering over those cracks, so giving rise to particles that are not only completely covered by that sealing shell, but also covered in a manner that enables the particles to be deagglomerated readily (e.g. using a non-aggressive technique, such as vortexing) in a manner that does not destroy the subshells that have been formed underneath, prior to, and/or during, pharmaceutical formulation.

For the reasons described herein, it is preferred that the sealing shell does not comprise zinc oxide. The sealing shell may on the other hand comprise silicon dioxide or, more preferably, aluminium oxide.

The second optional step may comprise ensuring that the few remaining particles with broken and/or cracked shells/coatings are subjected to a treatment in which all particles are suspended in a solvent in which the active ingredient is soluble (e.g. with a solubility of at least about 0.1 mg/mL), but the least soluble material in the mixed oxide coating is insoluble (e.g. with a solubility of no more than about 0.1 pg/mL), followed by separating solid matter particles from solvent by, for example, centrifugation, sedimentation, flocculation and/or filtration, resulting in mainly intact particles being left.

The above-mentioned optional step provides a means of potentially reducing further the likelihood of a (possibly) undesirable initial peak (burst) in plasma concentration of active ingredient, as discussed hereinbefore.

At the end of the process, coated particles may be dried using one or more of the techniques that are described hereinbefore for drying cores. Drying may take place in the absence, or in the presence, of one or more pharmaceutically-acceptable excipients (e.g. a sugar or a sugar alcohol).

Alternatively, at the end of the process, separated particles may be resuspended in a solvent (e.g. water, with or without the presence of one or more pharmaceutically acceptable excipients as defined herein), for subsequent storage and/or administration to patients.

Prior to applying the first layer of coating material or between successive coatings, cores and/or partially coated particles may be subjected to one or more alternative and/or preparatory surface treatments. In this respect, one or more intermediary layers comprising different materials (i.e. other than the inorganic material(s)) may be applied to the relevant surface, e.g. to protect the cores or partially-coated particles from unwanted reactions with precursors during the coating step(s)/deposition treatment, to enhance coating efficiency, or to reduce agglomeration.

An intermediary layer may, for example, comprise one or more surfactants, with a view to reducing agglomeration of particles to be coated and to provide a hydrophilic surface suitable for subsequent coatings. Suitable surfactants in this regard include well known non-ionic, anionic, cationic or zwitterionic surfactants, such as the Tween series, e.g. Tween 80. Alternatively, cores may be subjected to a preparatory surface treatment if the active ingredient that is employed as part of (or as) that core is susceptible to reaction with one or more precursor compounds that may be present in the gas phase during the coating (e.g. the ALD) process.

Application of ‘intermediary’ layers/surface treatments of this nature may alternatively be achieved by way of a liquid phase non-coating technique, followed by a lyophilisation, spray drying or other drying method, to provide particles with surface layers to which coating materials may be subsequently applied.

Outer surfaces of particles of formulations of the invention may also be derivatized or functionalized, e.g. by attachment of one or more chemical compounds or moieties to the outer surfaces of the final layer of coating material, e.g. with a compound or moiety that enhances the targeted delivery of the particles within a patient to whom the nanoparticles are administered. Such a compound may be an organic molecule (such as PEG) polymer, an antibody or antibody fragment, or a receptor-binding protein or peptide, etc.

Alternatively, the moiety may be an anchoring group such as a moiety comprising a silane function (see, for example, Herrera et al., J. Mater. Chem., 18, 3650 (2008) and U.S. Pat. No. 8,097,742). Another compound, e.g. a desired targeting compound may be attached to such an anchoring group by way of covalent bonding, or non-covalent bonding, including hydrogen bonding, or van der Waals bonding, or a combination thereof.

The presence of such anchoring groups may provide a versatile tool for targeted delivery to specific sites in the body. Alternatively, the use of compounds such as PEG may cause particles to circulate for a longer duration in the blood stream, ensuring that they do not become accumulated in the liver or the spleen (the natural mechanism by which the body eliminates particles, which may prevent delivery to diseased tissue).

According to one aspect of the invention, there is provided a plurality of coated particles obtained or obtainable by the method of preparing a plurality of coated particles according to the invention.

According to one aspect of the invention, there is provided a pharmaceutical or a veterinary composition, comprising a plurality of coated particles according to the invention and a pharmaceutically acceptable diluent, carrier and/or excipient. In one embodiment, said pharmaceutical composition is for human or animal use.

Such a pharmaceutical or a veterinary composition comprises a pharmacologically-effective amount of lenalidamide or pomalidomide or pharmaceutically-acceptable salt thereof. The term ‘pharmacologically-effective amount’ refers to an amount of lenalidamide or pomalidomide or salt thereof, which is capable of conferring a desired physiological change (such as a therapeutic effect) on a treated patient, whether administered alone or in combination with another active ingredient. Such a biological or medicinal response, or such an effect, in a patient may be subjective (i.e. the subject gives an indication of, or feels, an effect), and includes at least partial alleviation of the symptoms of the disease or disorder being treated, or curing or preventing said disease or disorder, or may be objective (i.e. measurable by some test or marker).

Pharmaceutical (or veterinary) formulations according to the invention may include particles of different types, for example particles comprising different functionalization (as described hereinbefore), particles of different sizes, and/or different thicknesses of the layers of mixed oxide coating materials, or a combination thereof. By combining, in a single pharmaceutical formulation, particles with different coating thicknesses and/or different core sizes, the drug release following administration to patient may be controlled (e.g. varied or extended) over a specific time period.

Dosages of active ingredient/salt thereof that may be administered to a patient should thus be sufficient to affect a therapeutic response over a reasonable and/or relevant timeframe. One skilled in the art will recognize that the selection of the exact dose and composition and the most appropriate delivery regimen will also be influenced by not only the nature of the active ingredient/salt, but also interalia the pharmacological properties of the formulation, the route of administration, the nature and severity of the condition being treated, and the physical condition and mental acuity of the recipient, as well as the age, condition, body weight, sex and response of the patient to be treated, and the stage/severity of the disease, as well as genetic differences between patients.

Dosages of active ingredient/salt thereof may also be determined by the timing and frequency of administration. In any event, the medical practitioner, or other skilled person, will be able to determine routinely the actual dosage of active ingredient, which will be most suitable for an individual patient.

Formulations of the invention may be administered systemically, for example by injection or infusion, intravenously or intraarterially (including by intravascular or other perivascular devices/dosage forms (e.g. stents)), intramuscularly, intraosseously, intracerebrally, intracerebroventricularly, intrasynovially, intrasternally, intrathecally, intralesionally, intracranially, intratumorally, cutaneously, intracutaneous, subcutaneously, transdermally, in the form of a pharmaceutically—(or veterinarily) acceptable dosage form. In particular embodiments, said formulation is administered intramuscularly or subcutaneously.

The preparation of formulations according to the invention comprises incorporation of coated particles of the invention into an appropriate pharmaceutically acceptable aqueous carrier system and may be achieved with due regard to the intended route of administration and standard pharmaceutical practice. Thus, appropriate excipients should be chemically inert to the active agent that is employed and have no detrimental side effects or toxicity under the conditions of use. Such pharmaceutically acceptable carriers may also impart an immediate, or a modified, release of the particles of the formulations according to the invention.

Sterile aqueous suspensions of the particles of the invention may be formulated according to techniques known in the art. The aqueous media should contain at least about 50% water, but may also comprise other aqueous excipients, such as Ringer's solution, and may also include polar co-solvents (e.g. ethanol, glycerol, propylene glycol, 1,3-butanediol, polyethylene glycols of various molecular weights and tetraglycol); viscosity-increasing, or thickening, agents (e.g. carboxymethylcellulose, microcrystalline cellulose, hydroxypropylmethyl cellulose, hydroxyethyl cellulose, ethyl hydroxyethyl cellulose, sodium starch glycolate, Poloxamers, such as Poloxamer 407, polyvinylpyrrolidone, cyclodextrins, such as hydroxypropyl-p-cyclodextrin, polyvinylpyrrolidone and polyethylene glycols of various molecular weights); surfactant/wetting agents to achieve a homogenous suspension (e.g. sorbitan esters, sodium lauryl sulfate; monoglycerides, polyoxyethylene esters, polyoxyethylene alkyl ethers, polyoxylglycerides and, preferably, Tweens (Polysorbates), such Tween 80 and Tween 20). Preferred ingredients include isotonicity-modifying agents (e.g. sodium lactate, dextrose and, especially, sodium chloride); pH adjusting and/or buffering agents (e.g. citric acid, sodium citrate, and especially phosphate buffers, such as disodium hydrogen phosphate dihydrate, sodium acid phosphate, sodium dihydrogen phosphate monohydrate and combinations thereof, which may be employed in combination with standard inorganic acids and bases, such as hydrochloric acid and sodium hydroxide); as well as other ingredients, such as mannitol, croscarmellose sodium and hyaluronic acid.

Formulations according to the invention may further be formulated in the form of injectable suspension of coated particles with a size distribution that is both even and capable of forming a stable suspension within the injection liquid (i.e. without settling) and may be injected through a needle. In this respect, the formulations of the invention may comprise an aqueous medium that comprises inactive ingredients that may prevent premature gelling of the formulations of the invention, and or is viscous enough to prevent sedimentation, leading to suspensions that are not ‘homogeneous’ and thus the risk of under or overdosing of active ingredient.

Formulations may thus be stored under normal storage conditions, and maintain their physical and/or chemical integrity. The phrase ‘maintaining physical and chemical integrity’ essentially means chemical stability and physical stability.

By ‘chemical stability’, we include that any formulation comprising coated particles of the invention may be stored (with or without appropriate pharmaceutical packaging), under normal storage conditions, with an insignificant degree of chemical degradation or decomposition.

By ‘physical stability’, we include that the any formulation comprising coated particles of the invention may be stored (with or without appropriate pharmaceutical packaging), under normal storage conditions, with an insignificant degree of physical transformation, such as sedimentation as described above, or changes in the nature and/or integrity of the coated particles, for example in the coating itself or the active ingredient (including dissolution, solvatisation, solid state phase transition, etc.).

Examples of ‘normal storage conditions’ for formulations of the invention include temperatures of between about −50° C. and about +80° C. (preferably between about −25° C. and about +75° C., such as about 500C), and/or pressures of between about 0.1 and about 2 bars (preferably atmospheric pressure), and/or exposure to about 460 lux of UV/visible light, and/or relative humidities of between about 5 and about 95% (preferably about 10 to about 40%), for prolonged periods (i.e. greater than or equal to about twelve, such as about six months).

Under such conditions, formulations of the invention may be found to be less than about 15%, more preferably less than about 10%, and especially less than about 5%, chemically and/or physically degraded/decomposed, as appropriate. The skilled person will appreciate that the above-mentioned upper and lower limits for temperature and pressure represent extremes of normal storage conditions, and that certain combinations of these extremes will not be experienced during normal storage (e.g. a temperature of 50° C. and a pressure of 0.1 bar).

Formulations of the invention may comprise between about 1% to about 99%, such as between about 10% (such as about 20%, e.g. about 50%) to about 90% by weight of the coated particles with the remainder made up by carrier system and/or other pharmaceutically-acceptable excipients.

According to a further embodiment of this aspect of the invention, the composition is in the form of a sterile injectable and/or infusible dosage form.

In any event, the preparation of suitable formulations may be achieved non-inventively by the skilled person using routine techniques. Formulations of the invention and dosage forms comprising them, may thus be formulated with conventional pharmaceutical additives and/or excipients used in the art for the preparation of pharmaceutical formulations, and thereafter incorporated into various kinds of pharmaceutical preparations and/or dosage forms using standard techniques (see, for example, Lachman et al, ‘The Theory and Practice of Industrial Pharmacy’, Lea & Febiger, 3rd edition (1986); ‘Remington: The Science and Practice of Pharmacy’, Troy (ed.), University of the Sciences in Philadelphia, 21st edition (2006); and/or ‘Aulton's Pharmaceutics: The Design and Manufacture of Medicines’, Aulton and Taylor (eds.), Elsevier, 4th edition, 2013), and the documents referred to therein, the relevant disclosures in all of which documents are hereby incorporated by reference.

According to an aspect of the invention, the coated particles are mixed with a carrier system after coating. According to a further aspect of the invention there is provided a process for the preparation of a formulation, which comprises mixing together the coated particles as described herein with an (e.g. aqueous) carrier system, for example as described herein. Such formulations are referred to hereinafter as ‘the formulations of the invention’.

For parenteral administration, such as subcutaneous and/or intramuscular injections, the formulations of the invention may be presented in the form of sterile injectable and/or infusible dosage forms administrable via a surgical administration apparatus (e.g. a syringe with a needle for injection, a catheter or the like), to form a depot formulation.

There is further provided an injectable and/or infusible dosage form comprising a formulation of the invention, wherein said formulation is contained within a reservoir that is connected to, and/or associated with, an injection or infusion means (e.g. a syringe with a needle for injection, a catherer or the like).

Alternatively, formulations of the invention can be stored prior to being loaded into a suitable injectable and/or infusible dosing means (e.g. a syringe with a needle for injection), or may even be prepared immediately prior to loading into such a dosing means.

Sterile injectable and/or infusible dosage forms may thus comprise a receptacle or a reservoir in communication with an injection or infusion means into which a formulation of the invention may be pre-loaded, or may be loaded at a point prior to use, or may comprise one or more reservoirs, within which coated particles of the formulation of the invention and the aqueous carrier system are housed separately, and in which admixing occurs prior to and/or during injection or infusion.

There is thus further provided a kit of parts comprising:
(a) coated particles of the formulation of the invention; and
(b) a carrier system of the formulation of the invention,
   as well as a kit of parts comprising coated particles of the formulation of the invention along with instructions to the end user to admix those particles with a carrier system according to the invention.

There is further provided a pre-loaded injectable and/or infusible dosage form as described herein above, but modified by comprising at least two chambers, within one of which chamber is located the coated particles of the formulation of the invention and within the other of which is located the aqueous carrier system of the formulation of the invention, wherein admixing, giving rise to a suspension or otherwise, occurs prior to and/or during injection or infusion.

According to one embodiment of this aspect of the invention, the composition is in the form of a liquid, a sol, or a gel, administrable via a surgical administration apparatus or a syringe, wherein the which liquid, sol, or gel forms a depot formulation in the patient, such as a subcutaneous depot formulation.

According to one embodiment of this aspect of the invention, the composition is in the form of a depot, or depot-forming, formulation releasing a therapeutically effective amount of said compound selected from lenalidomide and pomalidomide for a time period of 3 days to 3 months, such as 1 to 4 weeks, such as 15 to 25 days, such as approximately 3 weeks. In particular embodiments, the composition is in the form of a depot formulation releasing a therapeutically effective amount lenalidomide for a time period of 3 days to 3 months, such as 1 to 4 weeks, such as 15 to 25 days, such as approximately 3 weeks.

According to one embodiment of this aspect of the invention, said depot formulation is a subcutaneous or intramuscular depot, or depot-forming, formulation.

The composition may be intended for human patients and/or in human medicine. According to one aspect of the invention, there is provided a plurality of coated particles according to the invention or a pharmaceutical composition according to the invention as described herein for use as a medicament.

According to one aspect of the invention, there is provided a plurality of coated particles according to the invention or a pharmaceutical composition according to the invention for use in the treatment of hematologic cancers, such as for use in the treatment of multiple myeloma.

According to one embodiment of this aspect of the invention, the administration of said compound selected from the group consisting of lenalidomide and pomalidomide achieves an AUC of the compound of between about 10% and about 100%, such as about 10% and about 60% of the exposure (AUC) obtained from a standard of care treatment. In one embodiment, said AUC of the compound of between about 10% and about 60% of the exposure (AUC) obtained from a standard of care treatment is achieved by administration in the form a subcutaneous depot formulation as disclosed herein. The standard of care treatment can be intraperitoneal injection of, for example, about 500 pg once per day, or can be from about 2.5 to about 50 mg oral once daily, such as about 2.5 mg, about 4.0 mg, about 5 mg, about 10 mg, about 15 mg, about 20 mg, about 25 mg, about 30 mg, or about 50 mg of the immunomodulatory imide compound once daily or once every two days via oral administration. The standard of care treatment may also be an oral dose of lenalidomide from about 2.5 mg to about 50 mg once daily, such as at about 2.5 mg, about 5 mg, about 10 mg, about 15 mg, about 20 mg, or about 25 mg or about 50 mg once daily or once every two days.

According to one embodiment of this aspect of the invention, the total exposure to lenalidomide is at least about 40%, such as at least about 50%, such as at least about 60%, such as at least about 70%, such as at least about 80%, such as at least about 90%, such as up to about 100% of the total exposure obtained from a standard of care dosing regimen as defined above, for example a dosing regimen comprising orally administering about 25 mg or about 10 mg once daily over three consecutive days. In one embodiment, this is achieved by administration in the form a subcutaneous depot formulation as disclosed herein.

According to one embodiment of this aspect of the invention, the compound selected from the group consisting of lenalidomide and pomalidomide is continuously delivered at a rate of about 185 μg to about 725 pg/hour for treating newly diagnosed multiple myeloma. In one embodiment, this is achieved by administration in the form a subcutaneous depot formulation as disclosed herein.

According to one embodiment of this aspect of the invention, the administration of said compound selected from the group consisting of lenalidomide and pomalidomide achieves a steady state blood level of the compound in the range of about 19-70 ng/mL. In one embodiment, this is achieved by administration in the form a subcutaneous depot formulation as disclosed herein.

According to one embodiment of this aspect of the invention, the maximum concentration Cₘₐₓ of lenalidomide in plasma is at least about 40%, such as at least about 50%, such as at least about 60%, at least about 70%, such as at least about 80%, such as at least about 90%, such as up to about 100% of the Cₘₐₓ obtained from a dosing regimen comprising orally administering about 25 mg or about 10 mg once daily. The Cₘₐₓ obtained from a dosing regimen comprising orally administering an about 25 mg dose once daily is about 568 ng/ml. In one embodiment, this is achieved by administration in the form a subcutaneous depot formulation as disclosed herein.

According to one embodiment of this aspect of the invention, the compound selected from the group consisting of lenalidomide and pomalidomide is continuously delivered at a rate of about 70 μg to about 285 pg/hour for maintenance treatment of multiple myeloma. In one embodiment, this is achieved by administration in the form a subcutaneous depot formulation as disclosed herein.

According to one embodiment of this aspect of the invention, the administration of said compound selected from the group consisting of lenalidomide and pomalidomide achieves a steady state blood level of lenalidomide in the range of about 7.5 to about 28 ng/mL. In one embodiment, this is achieved by administration in the form a subcutaneous depot formulation as disclosed herein.

According to one embodiment of this aspect of the invention, the administration is in the form of a subcutaneous depot formulation.

The skilled person will appreciate that any embodiments mentioned in connection with the aspect related to the medical use of the particles as disclosed herein of the present disclosure are equally applicable to the inventive method of treatment as disclosed herein. For the sake of brevity, said embodiments will not be repeated here or will only be mentioned briefly.

According to one aspect of the invention, there is provided a method of treating hematologic cancers, such as multiple myeloma, comprising administering a therapeutically effective amount of a compound selected from the group consisting of lenalidomide and pomalidomide in the form of a plurality of coated particles according to the invention or in the form of a pharmaceutical composition according to the invention to a patient in need thereof.

According to one embodiment of this aspect of the invention, the compound is lenalidomide.

According to one embodiment of this aspect of the invention, the method achieves an AUC of said compound of between about 10% and about 60% of the exposure (AUC) obtained from a standard of care treatment. In one embodiment, this is achieved by administration in the form a subcutaneous depot formulation as disclosed herein.

According to one embodiment of this aspect of the invention, the method achieves a total exposure to lenalidomide is at least about 40%, such as at least about 50%, such as at least about 60%, such as at least about 70%, such as at least about 80%, such as at least about 90%, such as up to about 100% of the total exposure obtained from a dosing regimen comprising orally administering about 25 mg or about 10 mg once daily over three consecutive days. In one embodiment, this is achieved by administration in the form a subcutaneous depot formulation as disclosed herein.

According to one embodiment of this aspect of the invention, the compound is continuously delivered at a rate of about 185 μg to about 725 pg/hour for treating newly diagnosed multiple myeloma. In one embodiment, the administration in the form a subcutaneous depot formulation.

According to one embodiment of this aspect of the invention, the method achieves a steady state blood level of said compound in the range of about 19 to about 70 ng/mL. In one embodiment, this is achieved by administration in the form a subcutaneous depot formulation as disclosed herein.

According to one embodiment of this aspect of the invention, the method achieves a maximum concentration Cₘₐₓ of lenalidomide in plasma is at least about 40%, such as at least about 50%, such as at least about 60%, such as at least about 70%, such as at least about 80%, such as at least about 90%, such as up to about 100% of the Cₘₐₓ obtained from a dosing regimen comprising orally administering about 25 mg or about 10 mg once daily. The Cₘₐₓ obtained from a dosing regimen comprising orally administering an about 25 mg dose once daily is about 568 ng/mL. In one embodiment, this is achieved by administration in the form a subcutaneous depot formulation as disclosed herein.

According to one embodiment of this aspect of the invention, the compound is continuously delivered at a rate of about 70 μg to about 285 pg/hour for maintenance treatment of multiple myeloma. In one embodiment, the administration in the form a subcutaneous depot formulation.

According to one embodiment of this aspect of the invention, the method achieves a steady state blood level of said compound in the range of about 7.5 to about 28 pg/L. In one embodiment, this is achieved by administration in the form a subcutaneous depot formulation as disclosed herein.

According to one embodiment of this aspect of the invention, the administration is in the form of a subcutaneous or intramuscular depot, or depot-forming, formulation.

According to one embodiment of this aspect of the invention, the medicament is in the form and/or it forms a subcutaneous or intramuscular depot, or depot-forming, formulation.

According to the invention, there is provided a use of a plurality of coated particles according to the invention or a pharmaceutical composition according to the invention, in the preparation of a medicament for treating hematologic cancers. Typically, the medicament is for treating multiple myeloma.

Coated particles according to the invention, and formulations of the invention including them, are indicated in the therapeutic, palliative, and/or diagnostic treatment, as well as the prophylactic treatment (by which we include preventing and/or abrogating deterioration and/or worsening of a condition) of any of the above conditions.

In addition, injection of formulations of the invention may cause a mild inflammatory response. Such a response may be alleviated by co-administration with an antiinflammatory agent that is suitable for injection.

Appropriate antiinflammatory agents that may be employed in this regard include butylpyrazolidines (such as phenylbutazone, mofebutazone, oxyphenbutazone, clofezone, kebuzone and suxibuzone); acetic acid derivatives and related substances (indomethacin, sulindac, tolmetin, zomepirac, diclofenac, alclofenac, bumadizone, etodolac, lonazolac, fentiazac, acemetacin, difenpiramide, oxametacin, proglumetacin, ketorolac, aceclofenac and bufexamac); oxicams (such as piroxicam, tenoxicam, droxicam, lornoxicam and meloxicam); propionic acid derivatives (such as ibuprofen, naproxen, ketoprofen, fenoprofen, fenbufen, benoxaprofen, suprofen, pirprofen, flurbiprofen, indoprofen, tiaprofenic acid, oxaprozin, ibuproxam, dexibuprofen, flunoxaprofen, alminoprofen, dexketoprofen, vedaprofen, carprofen and tepoxalin); fenamates (such as mefenamic acid, tolfenamic acid, flufenamic acid, meclofenamic acid and flunixin), coxibs (such as celecoxib, rofecoxib, valdecoxib, parecoxib, etoricoxib, lumiracoxib, firocoxib, robenacoxib, mavacoxib and cimicoxib); other non-steroidal antiinflammatory agents (such as nabumetone, niflumic acid, azapropazone, glucosamine, benzydamine, glucosaminoglycan polysulfate, proquazone, orgotein, nimesulide, feprazone, diacerein, morniflumate, tenidap, oxaceprol, chondroitin sulfate, pentosan polysulfate and aminopropionitrile); corticosteroids (such as 11-dehydrocorticosterone, 11-deoxycorticosterone, 11-deoxycortisol, 11-ketoprogesterone, 11β-hydroxypregnenolone, 11β-hydroxyprogesterone, 11β,17α,21-trihydroxypregnenolone, 17α,21-dihydroxypregnenolone, 17α-hydroxypregnenolone, 17α-hydroxyprogesterone, 18-hydroxy-11-deoxycorticosterone, 18-hydroxycorticosterone, 18-hydroxyprogesterone, 21-deoxycortisol, 21-deoxycortisone, 21-hydroxypregnenolone (prebediolone), aldosterone, corticosterone (17-deoxycortisol), cortisol (hydrocortisone), cortisone, pregnenolone, progesterone, flugestone (flurogestone), fluorometholone, medrysone (hydroxymethylprogesterone), prebediolone acetate (21-acetoxypregnenolone), chloroprednisone, cloprednol, difluprednate, fludrocortisone, fluocinolone, fluperolone, fluprednisolone, loteprednol, methylprednisolone, prednicarbate, prednisolone, prednisone, tixocortol, triamcinolone, alclometasone, beclometasone, betamethasone, clobetasol, clobetasone, clocortolone, desoximetasone, dexamethasone, diflorasone, difluocortolone, fluclorolone, flumetasone, fluocortin, fluocortolone, fluprednidene, fluticasone, fluticasone furoate, halometasone, meprednisone, mometasone, mometasone furoate, paramethasone, prednylidene, rimexolone, ulobetasol (halobetasol), amcinonide, budesonide, ciclesonide, deflazacort, desonide, formocortal fluclorolone acetonide (flucloronide), fludroxycortide (flurandrenolone, flurandrenolide), flunisolide, fluocinolone acetonide, fluocinonide, halcinonide and triamcinolone acetonide); quinolines (such as oxycinchophen); gold preparations (such as sodium aurothiomalate, sodium aurothiosulfate, auranofin, aurothioglucose and aurotioprol); penicillamine and similar agents (such as bucillamine); and antihistamines (such as akrivastin, alimemazin, antazolin, astemizol, azatadin, azelastin, bamipin, bilastin, bromdifenhydramin, bromfeniramin, buklizin, cetirizin, cinnarizine, cyklizin, cyproheptadine, deptropine, desloratadin, dexbromfeniramin, dexklorfeniramin, difenylpyralin, dimenhydrinat, dimetinden, doxylamin, ebastin, epinastin, fenindamin, feniramin, fexofenadin, histapyrrodin, hydroxietylprometazin, isotipendyl, karbinoxamin, ketotifen, kifenadin, klemastin, klorcyklizin, klorfenamin, klorfenoxamin, kloropyramin, levocetirizin, loratadin, mebhydrolin, mekitazin, meklozin, mepyramin, metapyrilen, metdilazin, mizolastin, oxatomide, oxomemazine, pimetixen, prometazin, pyrrobutamin, rupatadin, sekifenadin, talastin, tenalidin, terfenadin, tiazinam, tietylperazin, tonzylamin, trimetobenzamid, tripelennamin, triprolidine and tritokvalin). Combinations of any one or more of the above mentioned antiinflammatory agents may be used.

Preferred antiinflammatory agents include non-steroidal anti-inflammatory drugs, such as diclofenac, ketoprofen, meloxicam, aceclofenac, flurbiprofen, parecoxib, ketoralac tromethamine or indomethacin.

Subjects may receive (or may already be receiving) one or more of the aforementioned antiinflammatory agents, separate to a formulation of the invention, by which we mean receiving a prescribed dose of one or more of those antiinflammatory agents, prior to, in addition to, and/or following, treatment with a formulation of the invention.

When lenalidomide or pomalidomide or salts of either are ‘combined’ with such other therapeutic agents, the active ingredients may be administered together in the same formulation, or administered separately (simultaneously or sequentially) in different formulations (hereinafter referred to as ‘combination products’).

Such combination products provide for the administration of lenalidomide/pomalidomide in conjunction with the other therapeutic agent, and may thus be presented either as separate formulations, wherein at least one of those formulations is a formulation of the invention, and at least one comprises the antiinflammatory agent in a separate formulation, or may be presented (i.e. formulated) as a combined preparation (i.e. presented as a single formulation including lenalidomide/pomalidomide/salt and the antiinflammatory agent).

In this respect an antiinflammatory agent may be co-presented with lenalidomide/pomalidomide at an appropriate dose in one or more of the cores that form part of a formulation of the invention as hereinbefore described, or may be formulated using the same or a similar process for coating to that described hereinbefore for lenalidomide/pomalidomide, which may allow for the release of the other therapeutic agent over the same, or over a different timescale.

Thus, there is further provided a pharmaceutical formulation of the invention that further comprises an antiinflammatory agent,

In such formulations of the invention, the further antiinflammatory agent may be included by:
(1) formulating along with the lenalidomide/pomalidomide within the solid cores of a formulation of the invention (which formulation is hereinafter referred to as a ‘combined core preparation’); or
(2) dissolving it, and/or suspending it, within the aqueous carrier system of a formulation of the invention (which formulation is hereinafter referred to as a ‘combination preparation’).

In embodiment (2) above, the antiinflammatory agent may be presented in a formulation of the invention in any form in which it is separate to the lenalidomide/pomalidomide-containing cores. This may be achieved by, for example, dissolving or suspending that active ingredient directly in the aqueous medium of a formulation of the invention, or by presenting it in a form in which its release can, like the lenalidomide/pomalidomide, also be controlled following injection.

The latter option may be achieved by, for example, providing the antiinflammatory agent in the form of additional particles suspended in the aqueous carrier system of formulation of the invention, which additional particles have a weight-, number-, or volume-based mean diameter that is between amount 10 nm and about 700 μm, and comprise cores comprising the antiinflammatory agent, which cores are coated, at least in part, by one or more coating materials as hereinbefore described (which formulation is hereinafter referred to as a ‘combination suspension’).

There is further provided a pharmaceutical formulation of the invention that is in the form of a kit of parts comprising components:
(A) a pharmaceutical formulation of the invention; and
(B) a pharmaceutical formulation, comprising an antiinflammatory agent,
which Components (A) and (B) are each provided in a form that is suitable for administration in conjunction with the other.

Although Component (B) of a kit of parts as presented above may be different in terms its chemical composition and/or physical form from Component (A) (i.e. a formulation of the invention), it may also be in a form that is essentially the same or at least similar to an lenalidomide/pomalidomide-containing formulation of the invention, that is in the form of a plurality of particles suspended in an (e.g. aqueous) carrier system, which particles:
(a) have a weight-, number-, or volume-based mean diameter that is between amount 10 nm and about 700 μm; and
(b) comprise solid cores comprising that other therapeutic agent, which cores are coated, at least in part, by one or more coatings of (e.g. inorganic) material.

In addition, although, in such preferred kits of parts, and the combination suspensions presented under embodiment (2) above, the coated cores comprising the other therapeutic agent may be different in terms of their chemical composition(s) and/or physical form(s), it is preferred that the coating of inorganic material that is employed is the same or similar to that employed in lenalidomide/pomalidomide-containing formulations of the invention, which means that the other therapeutic agent is coated by one or more inorganic coatings as hereinbefore described, for example one or more inorganic coating materials comprising one or more metal-containing, or metalloid-containing, compounds, such as a metal, or metalloid, oxide, for example iron oxide, titanium dioxide, zinc sulphide, more preferably zinc oxide, silicon dioxide and/or aluminium oxide, which coating materials may (on an individual or a collective basis) consist essentially (e.g. are greater than about 80%, such as greater than about, 90%, e.g. about 95%, such as about 98%) of such oxides, and more particularly inorganic coatings comprising a mixture of:
(i) zinc oxide; and
(ii) one or more other metal and/or metalloid oxides,
   wherein the atomic ratio ((i):(ii)) is at least about 1:6 and up to and including about 6:1.

Preferably, the atomic ratio ((i):(ii)) is at least about 1:1 and up to and including about 6:1.

In any event, and for the avoidance of doubt, all aspects, including preferred aspects, disclosed and/or claimed herein for in lenalidomide/pomalidomide-containing formulations of the invention are equally applicable as aspects and/or preferences for coated cores comprising one or more of the further aintiinflammatory agents described above. For the avoidance of doubt, such aspects, preferences and features, alone or in combination, are hereby incorporated by reference to these aspects of the invention.

All combination products, including combined core preparations, combination suspensions and kits of parts described above may thus be used in human medicine and, in particular, any indication in which lenalidomide and/or pomalidomide are either approved for use in, or otherwise known to be useful in, such as hematological cancers and multiple myeloma as hereinbefore defined.

According to a further aspect of the invention, there is provided a method of making a kit of parts as defined above, which method comprises bringing Component (A), as defined above, into association with a Component (B), as defined above, thus rendering the two components suitable for administration in conjunction with each other.

By bringing the two components ‘into association with’ each other, we include that Components (A) and (B) of the kit of parts may be:
(i) provided as separate formulations (i.e. independently of one another), which are subsequently brought together for use in conjunction with each other in combination treatment; or
(ii) packaged and presented together as separate components of a ‘combination pack’ for use in conjunction with each other in combination treatment.

Thus, there is further provided a kit of parts as hereinbefore defined in which Components (A) and (B) are packaged and presented together as separate components of a combination pack, for use in conjunction with each other in combination treatment, as well as a kit of parts comprising:
(I) one of Components (A) and (B) as defined herein; together with
(II) instructions to use that component in conjunction with the other of the two components.

As alluded to above, the kits of parts described herein may comprise more than one formulation including an appropriate quantity/dose of lenalidomide/pomalidomide/salt, and/or more than one formulation including an appropriate quantity/dose of the antiinflammatory agent, in order to provide for repeat dosing as hereinbefore described.

In this respect, with respect to the kits of parts as described herein, by ‘administration in conjunction with’, we include that Components (A) and (B) of the kit are administered, sequentially, separately and/or simultaneously, over the course of treatment of the condition.

Thus, the term ‘in conjunction with’ includes that one or other of the two formulations may be administered (optionally repeatedly) prior to, after, and/or at the same time as, administration of the other component. When used in this context, the terms ‘administered simultaneously’ and ‘administered at the same time as’ include that individual doses of lenalidomide/pomalidomide/salt and antiinflammatory agent are administered within 48 hours (e.g. 24 hours) of each other.

In respect of any of the above combination products according to the invention, the respective formulations are administered (or, in the case of the kit of parts, the two components are administered, optionally repeatedly, in conjunction with each other) in a manner that may enable a beneficial effect for the subject, that is greater, over the course of the treatment of the condition, than if a formulation (e.g. a formulation of the invention) comprising lenalidomide/pomalidomide/salt alone is administered (e.g. repeatedly, as described herein) in the absence of the other component, over the same course of treatment.

Determination of whether a combination product provides a greater beneficial effect in respect of, and over the course of treatment will depend upon the condition to be treated and/or its severity, but may be achieved routinely by the skilled person.

For example, a physician may initially administer a formulation of the invention comprising alone to treat a patient with multiple myeloma, and then find that that person exhibits an inflammatory response (which may be caused by the active ingredient per se and/or by any other component of the formulation).

The physician may then administer one or more of:
Component (B) of a kit of parts as described above,
a combined core preparation,
a combination preparation, and/or
a combination suspension
   as described above, any of which comprises an antiinflammatory agent as hereinbefore described.

The antiinflammatory agents mentioned above that may be employed in combination products according to the invention may be provided in the form of a (e.g. pharmaceutically-acceptable) salt, including any such salts that are known in the art and described for the drugs in question to in the medical literature, such as Martindale—The Complete Drug Reference, 38^{th} Edition, Pharmaceutical Press, London (2014) and the documents referred to therein (the relevant disclosures in all of which documents are hereby incorporated by reference).

The amount of the antiinflammatory agent that may be employed in combination products according to the invention must be sufficient so exert its pharmacological effect.

Doses of such aintiflammatory ingredients that may be administered to a patient should thus be sufficient to affect a therapeutic response over a reasonable and/or relevant timeframe. One skilled in the art will recognize that the selection of the exact dose and composition and the most appropriate delivery regimen will also be influenced by not only the nature of the other antiinflammatory ingredient, but also inter alia the pharmacological properties of the formulation, the route of administration, the nature and severity of the condition being treated, and the physical condition and mental acuity of the recipient, as well as the age, condition, body weight, sex and response of the patient to be treated, and the stage/severity of the disease, as well as genetic differences between patients.

As administration of formulations of the invention may be continuous or intermittent (e.g. by bolus injection), dosages of the antiinflammatory ingredients may also be determined by the timing and frequency of administration.

In any event, the medical practitioner, or other skilled person, will be able to determine routinely the actual dosage of any particular additional active ingredient, which will be most suitable for an individual patient, and doses of the relevant additional active ingredients mentioned above include those that are known in the art and described for the drugs in question to in the medical literature, such as Martindale—The Complete Drug Reference, 38^{th} Edition, Pharmaceutical Press, London (2014) and the documents referred to therein, the relevant disclosures in all of which documents are hereby incorporated by reference.

The use of coated particles according to the invention, and formulations of the invention including them, may control the dissolution rate of active ingredient and affect the pharmacokinetic profile by reducing any burst effect as hereinbefore defined and/or by reducing Cₘₐₓ in a plasma concentration-time profile.

Coated particles according to the invention, and formulations of the invention including them, may also provide a release and/or pharmacokinetic profile that increases the length of release of active ingredient from the formulation.

These factors not only reduce the frequency at or over which a formulation needs to be administered to a multiple myeloma sufferer, but also allows the sufferer more time as an out-patient, and so to have a better quality of life.

Coated particles according to the invention, and formulations of the invention including them, also have the advantage that by controlling the release of active ingredient at a steady rate over a prolonged period of time, a lower daily exposure to cytotoxic drug is needed, which is expected to reduce unwanted side effects.

The coated particles, formulations and processes described herein may also have the advantage that, in the treatment of the relevant conditions, they may be more convenient for the physician and/or patient than, be more efficacious than, be less toxic than, have a broader range of activity than, be more potent than, produce fewer side effects than, or that it may have other useful pharmacological properties over, any similar treatments known in the prior art.

Wherever the word ‘about’ is employed herein, for example in the context of amounts (e.g. numbers, concentrations, dimensions (sizes and/or weights), doses, time periods, pharmacokinetic parameters, etc.), relative amounts (percentages, weight ratios, size ratios, atomic ratios, aspect ratios, proportions, factors or fractions), temperatures, relative humidities, lux or pressures, it will be appreciated that such variables are approximate and as such may vary by ±15%, such as ±10%, for example ±5% and preferably ±2% (e.g. ±1%) from the numbers specified herein. This is the case even if such numbers are presented as percentages in the first place (for example ‘about 15%’ may mean±15% about the number 10, which is anything between 8.5% and 11.5%).

### Items of the Invention

1. A plurality of coated particles comprising:
(a) a solid core comprising a compound selected from the group consisting of lenalidomide and pomalidomide, or a pharmaceutically acceptable salt thereof, said core having a mean diameter of the between 0.1 μm and 50 μm; and
(b) a coating surrounding, enclosing and/or encapsulating said core, wherein the coating comprises a mixed oxide of at least two metal or metalloid oxides selected from the group consisting of iron oxide, zinc oxide, silicon oxide, titanium oxide, and aluminium oxide,
wherein the particles may be useful in the treatment of hematologic cancers, such as multiple myeloma.
2. The plurality of coated particles according to item 1, wherein said compound is lenalidomide.
3. The plurality of coated particles according to item 1 or 2, wherein said coating comprises 1 to 20 discrete layers, wherein each layer comprises at least one oxide selected from the group consisting of iron oxide, zinc oxide, silicon oxide, titanium oxide, and aluminium oxide, and wherein at least one out of said 1 to 20 discrete layers comprises said mixed oxide.
4. The plurality of coated particles according to any one of items 1 to 3, wherein said coating comprises 3 to 7 discrete layers.
5. The plurality of coated particles according to any one of items 1 to 4, wherein said coating comprises 4 to 6 layers discrete layers.
6. The plurality of coated particles according to any one of items 3 to 5, wherein at least 2, such as at least 3, of said discrete layers comprise said mixed oxide.
7. The plurality of coated particles according to any one of items 3 to 6, wherein at least 4 of said discrete layers comprise said mixed oxide.
8. The plurality of coated particles according to any one of items 3 to 7, wherein all of said discrete layers comprise said mixed oxide.
9. The plurality of coated particles according to any one of items 3 to 8, wherein said at least one oxide is selected from the group consisting of zinc oxide, silicon oxide, and aluminium oxide.
10. The plurality of coated particles according to any one of items 3 to 9, wherein said at least one oxide is zinc oxide or aluminium oxide.
11. The plurality of coated particles according to any one of items 3 to 9, wherein said at least one oxide is zinc oxide or silicon oxide.
12. The plurality of coated particles according to any one of items 1 to 11, wherein said at least two metal and/or metalloid oxides in said mixed oxide are selected from the group consisting of zinc oxide, silicon oxide, and aluminium oxide.
13. The plurality of coated particles according to any one of items 1 to 12, wherein said at least two metal and/or metalloid oxides in said mixed oxide are zinc oxide and aluminium oxide.
14. The plurality of coated particles according to any one of items 1 to 12, wherein said at least two metal and/or metalloid oxides in said mixed oxide are zinc oxide and silicon oxide.
15. The plurality of coated particles according to item 13, wherein the atomic ratio of Zn to Al is between 1 to 1 and 6 to 1.
16. The plurality of coated particles according to item 13 or 15, wherein the atomic ratio of Zn to Al is between 2 to 1 and 5 to 1.
17. The plurality of coated particles according to item 13, 15 or 16, wherein the atomic ratio of Zn to Al is between 2 to 1 and 4 to 1.
18. The plurality of coated particles according to item 13 or any one of 15 to 17, wherein the atomic ratio of Zn to Al is 3 to 1.
19. The plurality of coated particles according to any one of items 1 to 18, wherein the total thickness of the coating is between about 0.2 nm and about 2 μm.
20. The plurality of coated particles according to any one of items 1 to 19, wherein said core has a mean diameter of the between 3 μm and 20 μm, such as 5 μm to 15 μm, such as 8 μm to 12 μm, such as 10 μm.
21. A method of preparing a plurality of coated particles according to any one of items 1 to 20, wherein the coated particles are made by applying the precursors of at least two metal and/or metal oxides forming a mixed oxide on the solid cores, and/or the previously-coated solid cores, by a gas phase deposition technique.
22. A method of according to item 21, said method comprising the steps of:
(1) applying precursors of one or more metal and/or metalloid oxides selected from the group consisting of iron oxide, zinc oxide, silicon oxide, titanium oxide, and aluminium oxide, whereby a layer is formed on said solid cores by way of a gas phase deposition technique;
(2) disaggregating the coated solid core aggregates formed during step (1);
(3) applying precursors of at least one metal and/or metalloid oxides selected from the group consisting of iron oxide, zinc oxide, silicon oxide, titanium oxide, and aluminium oxide to the disaggregated, coated solid cores from step (2) whereby another layer is formed on said previously-coated solid cores by way of a gas phase deposition technique; and
(4) repeating steps (2) and (3) one or more times to increase the total thickness of the coating that encloses said solid core,
wherein at least one of the layers is formed by applying precursors of at least two metal and/or metalloid oxides selected from the group consisting of iron oxide, zinc oxide, silicon oxide, titanium oxide, and aluminium oxide, whereby a mixed oxide is formed.
23. The method according to item 22, wherein said disaggregating coated particles aggregates is done by way of a mechanical sieving technique.
24. The method according to any one of items 22 to 23, wherein step 2 further comprises a step of discharging the coated solid cores from the gas phase deposition reactor, prior to disaggregating coated solid core aggregates formed during step (1); and the step of charging the coated solid cores back to the reactor subsequent to disaggregating coated solid cores aggregates formed during step (1).
25. The method according to any one of items 21 to 24, wherein the gas phase deposition technique is selected from atomic layer deposition, chemical vapour deposition, and physical vapour deposition.
26. The method according to any one of items 21 to 25, wherein the gas phase deposition technique is atomic layer deposition.
27. The method according to any one of items 21 to 26, wherein said precursors are:
an oxygen containing precursor, such as water, oxygen, ozone and/or hydrogen peroxide; and
a metal and/or metalloid compound selected from diethylzinc, trimethylaluminium, and orthosilicic acid tetraethyl ester.
28. The method according to any one of items 23 to 27, wherein the mechanical sieving technique is sonic sifting or vibrational sieving.
29. The method according to item 28, wherein the mechanical sieving technique is sonic sifting.
30. The method according to item 28, wherein the mechanical sieving technique is vibrational sieving.
31. A pharmaceutical or veterinary composition, comprising plurality of coated particles according to any one of items 1 to 20 and a pharmaceutically acceptable diluent, carrier and/or excipient.
32. The composition according to item 31 in the form of a sterile injectable and/or infusible dosage form.
33. The composition according to item 31 or 32, wherein the coated particles are mixed with a carrier system after coating.
34. The composition according to any one of items 31 to 33, wherein the composition comprises optionally crosslinked hyaluronic acid.
35. The composition according to any one of items 31 to 34 in the form of a liquid, a sol, or a gel, administrable via a surgical administration apparatus or a syringe, which liquid, sol, or gel forms a depot formulation.
36. The composition according to item 35, wherein the composition is in the form of a depot formulation releasing a therapeutically effective amount of said compound selected from lenalidomide and pomalidomide for a time period of 3 days to 3 months, such as 1 to 4 weeks, such as 15 to 25 days, such as approximately 3 weeks.
37. The composition according to items 35 or 36, wherein said depot formulation is a subcutaneous depot formulation.
38. A plurality of coated particles according to any one of items 1 to 20 or pharmaceutical composition according to any one of items 31 to 37 for use as a medicament.
39. A plurality of coated particles according to any one of items 1 to 20 or pharmaceutical composition according to any one of items 31 to 37 for use in the treatment of hematologic cancers, such as for use in the treatment of multiple myeloma.
40. The plurality of coated particles for use or pharmaceutical composition for use according to item 38 or 39, wherein the administration of said compound selected from the group consisting of lenalidomide and pomalidomide achieves an AUC of the compound of between 10% and 100% of the exposure (AUC) obtained from a standard of care treatment.
41. The plurality of coated particles for use or pharmaceutical composition for use according to item 38 or 39, wherein the total exposure to lenalidomide is at least about 40%, such as at least 50%, such as at least 60%, of the total exposure obtained from a dosing regimen comprising orally administering 25 mg or 10 mg once daily over three consecutive days.
42. The plurality of coated particles or pharmaceutical composition for use according to item 38 or 39, wherein said compound selected from the group consisting of lenalidomide and pomalidomide is continuously delivered at a rate of 185 μg to 725 pg/hour for treating newly diagnosed multiple myeloma.
43. The plurality of coated particles or pharmaceutical composition for use according to item 38 or 39, wherein the administration of said compound selected from the group consisting of lenalidomide and pomalidomide achieves a steady state blood level of the compound in the range of about 19-70 pg/L.
44. The plurality of coated particles or pharmaceutical composition for use according to item 38 or 39, wherein the maximum concentration Cₘₐₓ of lenalidomide in plasma is at least about 40%, such as at least 50%, such as at least 60%, of the Cₘₐₓ obtained from a dosing regimen comprising orally administering 25 mg or 10 mg once daily.
45. The plurality of coated particles or pharmaceutical composition for use according to item 38 or 39, wherein said compound selected from the group consisting of lenalidomide and pomalidomide is continuously delivered at a rate of 70 μg to 285 pg/hour for maintenance treatment of multiple myeloma.
46. The plurality of coated particles or pharmaceutical composition for use according to item 38 or 39, wherein the administration of said compound selected from the group consisting of lenalidomide and pomalidomide achieves a steady state blood level of lenalidomide in the range of about 7.5-28 pg/L.
47. The plurality of coated particles or pharmaceutical composition for use according to any one of items 40-46, wherein said administration is in the form of a subcutaneous depot formulation.
48. A method of treating hematologic cancers, such as multiple myeloma, comprising administering a therapeutically effective amount of a compound selected from the group consisting of lenalidomide and pomalidomide in the form of a plurality of coated particles according to any one of items 1 to 20 or in the form of a pharmaceutical composition according to any one of items 31 to 37 to a patient in need thereof.
49. The method according to item 48, wherein said compound is lenalidomide.
50. The method according to item 48 or 49, wherein the method achieves an AUC of said compound of between 10% and 60% of the exposure (AUC) obtained from a standard of care treatment.
51. The method according to item 48 or 49, wherein the method achieves a total exposure to lenalidomide is at least about 40%, such as at least 50%, such as at least 60%, of the total exposure obtained from a dosing regimen comprising orally administering 25 mg or 10 mg once daily over three consecutive days.
52. The method according to item 48 or 49, wherein said compound is continuously delivered at a rate of 185 μg to 725 pg/hour for treating newly diagnosed multiple myeloma.
53. The method according to item 48 or 49, wherein the method achieves a steady state blood level of said compound in the range of about 19-70 pg/L.
54. The method according to item 48 or 49, wherein the method achieves a maximum concentration Cₘₐₓ of lenalidomide in plasma is at least about 40%, such as at least 50%, such as at least 60%, of the Cₘₐₓ obtained from a dosing regimen comprising orally administering 25 mg or 10 mg once daily.
55. The method according to item 48 or 49, wherein said is continuously delivered at a rate of 70 μg to 285 pg/hour for maintenance treatment of multiple myeloma.
56. The method according to item 48 or 49, wherein the method achieves a steady state blood level of said compound in the range of about 7.5-28 pg/L.
57. The method according to any one of items 48 to 56, wherein said administration is in the form of a subcutaneous depot formulation.
58. Use of a plurality of coated particles according to any one of items 1 to 20 or a pharmaceutical composition according to any one of items 31 to 37, in the preparation of a medicament for treating hematologic cancers.
59. Use according to item 58, wherein the medicament is for treating multiple myeloma.
60. Use according to item 58 or 59, wherein said medicament is in the form a subcutaneous depot formulation.

While the invention has been described with reference to various exemplary aspects and embodiments, it will be understood by those skilled in the art that various changes may be made and equivalents may be substituted for elements thereof without departing from the scope of the invention. Therefore, it is intended that the invention not be limited to any particular embodiment contemplated, but that the invention will include all embodiments falling within the scope of the appended claims. The invention will be further illustrated by the following non-limiting Examples.

### BRIEF DESCRIPTION OF THE FIGURES

FIG. 1 is a graph showing the in vitro release profile of lenalidomide from uncoated particles according to Comparative Example 3.

FIG. 2 is a graph showing the in vitro release profile of lenalidomide coated with four distinct layers according to Example 1.

FIG. 3 is a graph showing the in vitro release profile of lenalidomide coated with six distinct layers according to Example 2.

FIG. 4 shows transmission electromicroscopy images of particles with a coating produced using a process comprising a deagglomeration step. Arrows indicate visible interphases between atomic layers applied.

FIG. 5(a) and 5(b) show dose-normalised plasma concentrations of azacytidine (azacitidine) over time after subcutaneous administration to rats of various formulations as described in Example 14.

FIGS. 6 to 9 show dose-adjusted plasma concentrations (ng/mL) over time (h) curves following subcutaneous administration of coated Ienolidamide formulations prepared according Example 21 to rats (n=6). In particular, FIG. 6 represents a formulation including the coated particles of Comparative Example 15 (50 mg/mL; light grey triangles), FIG. 7 represents formulations including the coated particles of Comparative Example 16 (5 mg/mL (dark grey squares) and 50 mg/mL (light grey circles)), FIG. 8 represents a formulation including the coated particles of Example 17 (10 mg/mL; black hexagons) and FIG. 9 represents a formulation including the coated particles of Example 18 (10 mg/mL; black diamonds). In all cases, error bars indicate standard error.

### EXAMPLES

### Example 1—Production of Coated Particles According to the Invention

The present example describes the process of production of coated lenalidomide particles with four distinct coating layers according to the present invention.

### Material and Method

Microparticles of lenalidomide Form I, 99.9% were used obtained from APIChem, China, and were used as received. The mean diameter of the lenalidomide particles was 10 μm as determined by laser diffraction (Shimadzu, SALD-7500nano, Kyoto, Japan). The particle size distribution, as determined by laser diffraction, was as follows: D₁₀ 2.7 μm; D₅₀ 10.4 μm and D₉₀ 24.9 μm.

The microparticles were coated as described in steps 1-4. 1. The powder was loaded to an ALD reactor (Picosun, SUNALE™ R-series, Espoo, Finland) and was subjected to ALD cycles. Three ALD cycles employing diethyl zinc and water as precursors, followed by one cycle of trimethylaluminium and water as precursors at a reactor temperature of 50° C. This was repeated six times, i.e. 6×(3 cycles of Zn+1 cycle of Al). This way a first layer of mixed oxide with a atomic ratio of zinc:aluminium 3:1 was formed.

The ALD-cycle was performed as follows (steps a-e represent the first cycle, subsequent cycles start from step b as specified in step f and the last cycle ends at step e):

Reagent pulse: Water was evaporated and carried into the reaction chamber by inert nitrogen gas. Water adsorb to the surface of the drug particles, presenting hydroxyl groups on the exterior or the particle.

Purging pulse: The chamber was purged with nitrogen. Gaseous water, and organic gases in case this is not the first cycle, are removed.

Reagent pulse: Diethylzinc or trimethylaluminum was evaporated and carried into the reaction chamber by inert nitrogen gas. Diethylzinc or trimethylaluminum adsorbs to the surface of the drug particles and reacts with hydroxyl groups. This releases ethane from diethylzinc or methane from trimethylaluminum.

Purging pulse: The chamber was purged with nitrogen. Non-reacted reagents and organic gases are removed.

Reagent pulse: Water was evaporated and carried into the reaction chamber by inert nitrogen gas. Water adsorb to the surface of the drug particles and reacts with the metalloorganic surface. All remaining ethyl or methyl groups are converted to ethane and methane, respectively. The surface is thereby covered in a metal oxide layer, presenting hydroxyl groups on the exterior of the particle.

The cycle was repeated from step b.
2. Next the powder was removed from the reactor and deagglomerated by means of sonic sifter (Tsutsui Sonic Agitated Sifting Machine SW-20AT) with a 32 μm mesh size sieve.
3. The resultant deagglomerated powder was re-loaded into the ALD reactor and steps 1-2 were repeated two times, forming a second and a third layer of mixed oxide with a atomic ratio of approximately zinc:aluminium 3:1.
4. The resultant deagglomerated powder was re-loaded into the ALD reactor and step 1 was repeated one time.

### Results

The obtained particles had four layers of mixed oxide with a atomic ratio of zinc atoms:aluminium atoms of approximately 3:1.

Depending on the number of times the steps 1-3 are repeated, particles with different numbers of distinct layers may be obtained. Thus, the cycles are repeated until the desired amount of mixed oxide coating on the drug particles is achieved. Particles with at least one and up to ten discrete layers will be produced.

### Example 2—Production of Coated Particles According to the Invention

The present example describes the process of production of coated lenalidomide particles with six distinct coating layers according to the present invention is described.

### Material and Method

Microparticles of lenalidomide Form I (APIChem, China), 99.9% were used as received as described in Example 1.

The microparticles were coated as described in steps 1-4 and the ALD-cycles were performed as described in Example 1.
1. The powder was loaded to an ALD reactor (Picosun, SUNALE™ R-series, Espoo, Finland). Three ALD cycles employing diethyl zinc and water as precursors, followed by one cycle of trimethylaluminium and water as precursors at a reactor temperature of 50° C. This was repeated six times, i.e. 6×(3 cycles of Zn+1 cycle of Al). This way a first layer of mixed oxide with a atomic ratio of approximately zinc:aluminium 3:1 was formed.
2. The powder was removed from the reactor and deagglomerated by means of sonic sifter (Tsutsui Sonic Agitated Sifting Machine SW-20AT) with a 32 μm mesh size sieve.
3. The resultant deagglomerated powder was re-loaded into the ALD reactor and steps 1-2 were repeated four times, forming a second, third, fourth and a fifth layer of mixed oxide with a atomic ratio of zinc:aluminium 3:1.
4. The resultant deagglomerated powder was re-loaded into the ALD reactor and step 1 was repeated one time.

### Results

The obtained particles had six layers of mixed oxide with a atomic ratio of zinc atoms:aluminium atoms of approximately 3:1 was formed.

### Comparative Example 3—Lenalidomide Microparticles without Coating

Particles according to Comparative Example 3 are the microparticles of lenalidomide Form I, 99.9% which were obtained from APIChem, China, as described above for Example 1 and 2. The Comparative Example 3 particles have not been subject to any coating.

### Comparative Example 4—Aluminium Oxide Coated Lenalidomide Microparticles

The same microparticles of lenalidomide Form I, 99.9% which obtained from APIChem, China, as described above for Example 1 and 2 are coated a pure aluminium oxide coating. The coating is performed as described in Example 1, however seven ALD cycles are performed prior to removing the coated powder from the reactor and deagglomerated as described in Example 1. The resultant deagglomerated powder is reloaded into the ALD reactor and subjected to a further 7 ALD cycles, followed by extraction, deagglomeration and reloading for 7 cycles was repeated twice thereafter, followed by reloading and subjecting to 2 times 14 cycles.

The drug load of said particles will be determined as described in Example 6 below.

### Comparative Example 5—Zinc Oxide Coated Lenalidomide Microparticles

The same microparticles of lenalidomide Form I, 99.9% which obtained from APIChem, China, as described above for Example 1 and 2 are coated a pure zinc oxide coating. The coating is performed as described in Example 1, however seven ALD cycles are performed prior to removing the coated powder from the reactor and deagglomerated as described in Example 1. The resultant deagglomerated powder is reloaded into the ALD reactor and subjected to a further 7 ALD cycles, followed by extraction, deagglomeration and reloading for 7 cycles was repeated twice thereafter, followed by reloading and subjecting to 2 times 14 cycles.

The drug load of said particles will be determined as described in Example 6 below.

### Example 6—Determination of Drug Load

The present example describes the analysis of the drug load of lenalidomide of the particles obtained in Example 1 and 2.

### Material and Method

To determine the drug load (i.e. w/w % of lenalidomide in the powder), UPLC (Prominence-i (Shimadzu, Japan) equipped with a diode array detector (Shimadzu, Japan) set at 223 nm was employed using a 4.6×100 mm, 2.6 μm particles, C18WP column (SunShell, ChromaNik Technologies Inc, Osaka, Japan)) was used. The materials were dissolved in 2 M phosphoric acid in acetonitril/water (1:1) and was diluted with methanol/12.5 mM phosphate buffer pH 3.6 (4:1) before filtration (0.2 μm RC, Lab Logistics Group, Germany) and further analyzed with UPLC (n=2).

The UPLC assay is set-up according to

TABLE 1
Parameters for the UPLC-method.

| Column | SunShell C18WP 4.6*100 mm, | |
|---|---|---|
| | 2.6 μm particles, Nanexa column ID 1. | |
| Temperature, column oven | 27° C. | |
| Flow | 0.7 mL/min | |
| Gradient program | Time (min) | % Mobile phase B |
| | 0-0.5 | 10 |
| | 0.5-16 | 70 |
| | 16-16.10 | 10 |
| | 16.10-19 | 10 |

| | |
|---|---|
| Mobile phase A | 12.5 mM phosphate buffer pH 3.6 |
| Mobile phase B | Methanol |
| Wash solution | 80% Acetonitrile in water |
| Run time | 19 min |
| Temperature, autosampler | 25° C. |
| Injection volume | 5 μL |
| UV detection wavelength | 223 nm |

Concentrations of injected samples are calculated automatically by the software supplied by the manufacturer. Drug load was calculated according to the equation below, wherein C_{LEN} is the analyzed concentration of lenalidomide and mₛₐₘₚₗₑ is sample mass

$$Drugload ( \% ) = 100 \times \frac{{C}_{LEN} ( mg / L )}{\frac{{m}_{sample} ( mg )}{End Volume ( L )}}$$

### Results

The drug load lenalidomide of the particles according to Example 1 was determined as 94.8%.

The drug load lenalidomide of the particles according to Example 2 was determined as 92.6%.

### Example 7—Analysis of In Vitro Drug Release

The present example describes the analysis of the in vitro drug release of lenalidomide from the particles obtained in Example 1 and 2 and from particles according to Comparative Example 3.

### Material and Method

In vitro release studies for the particles of Comparative Example 3 and Example 1 were conducted using a Sotax CE 7smart USP 4 apparatus (Sotax AG, Switzerland) linked to a CP 7-35 piston pump (Sotax AG, Switzerland) and a C613 fraction collector (Sotax AG, Switzerland).

Flow-through cells with a 22.6 mm diameter were prepared with a 5 mm ruby bead in the tip of the cell cone, in which the suspended samples were introduced.

The samples were analyzed in duplicates with a sample amount corresponding to 50 mg lenalidomide per cell. The samples (33.3 mg lenalidomide/mL) were dispersed by vortexing in 1% poloxamer P188 in 20 mM PIPES buffer with a pH of 7.2.

The apparatus was used in an open-loop set-up, in which fresh 20 mM PIPES buffer, pH 7.2 dissolution medium was continuously introduced into the system. The temperature of the water bath was set at 37° C. t 0.5° C. and the flow rate of media was set at 16 mL/min. The medium was filtered before leaving the flow through cells using two Whatman glass microfiber filters, GF/F and GF/D (d=25 mm, Sigma-Aldrich/Merck KGaA, Germany). The collected fractions of the release medium were analyzed for lenalidomide content using UV-VIS (Shimadzu UV-1800 spectrophotometer equipped with a sipper unit) at 244 nm.

### Results

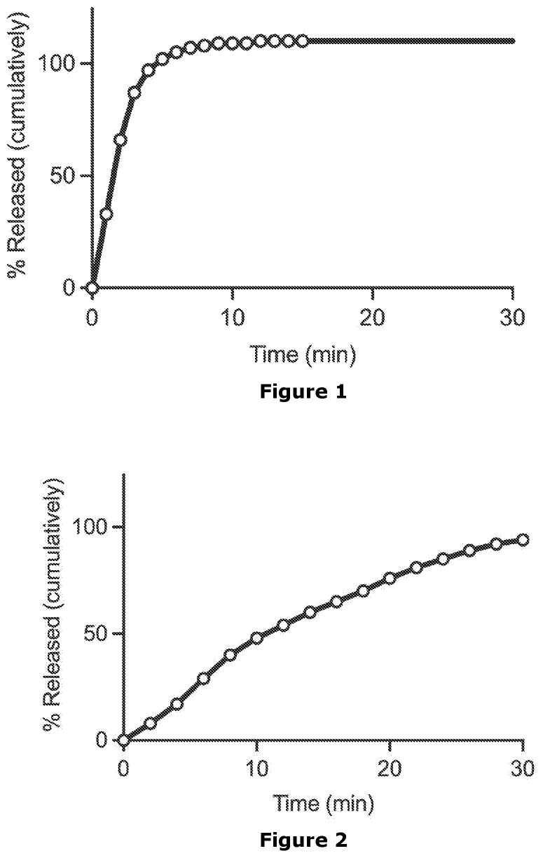

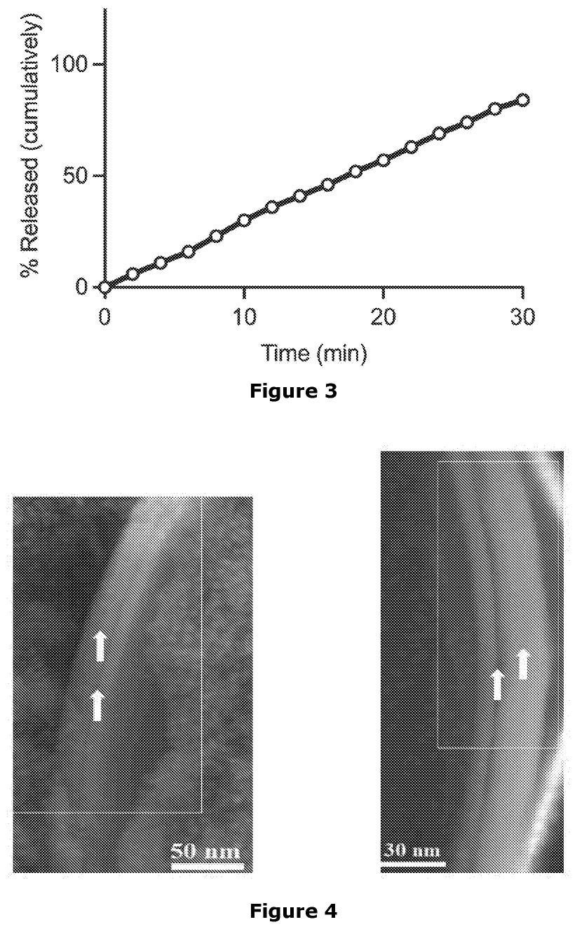

FIGS. 1, 2 and 3 show the respective lenalidomide release profiles (cumulative percentage of lenalidomide released versus sampling time in the Sotax apparatus for samples obtained by Comparative Example 3, Example 1 and Example 2, respectively. This example demonstrated that the coated lenalidomide particles as described herein, exhibited a sustained release of lenalidomide compared with uncoated lenalidomide particles (Comparative Example 3).

### Example 8—Determination of Particle Size Distribution

The present example describes the determination of particle size distribution of the particles according to Example 1, 2 and Comparative Example 3

### Material and Method

The particle size distribution of the particles was investigated by laser diffraction (Shimadzu, SALD-7500nano, Kyoto, Japan) according to the manufacturer's instructions.

### Results

The uncoated particles had a mean diameter of 10 μm and the coated particles according to Example 1 and 2 had a mean diameter of 17.7 (D50) and 15.9 (D50) μm, respectively.

### Example 9—Preparation of Formulations

The present example describes the preparation of formulations of the coated lenalidomide particles as disclosed herein.

### Material and Method

A suspension of coated microparticles of lenalidomide according to Example 1 and 2 and Comparative Example 3 is prepared.

The powder of coated microparticles of lenalidomide according to Example 1 and 2 and Comparative Example 3 is mixed together with Hyonate® vet (Boehringer Ingelheim Animal Health, France) as vehicle. Hyonate® vet is a veterinary medicinal product used in injecting animals comprising a sterile, isotonic, phosphate buffered solution of 10 mg/mL of sodium hyaluronate (pH 7.4). The composition of Hyonate® vet is a presented in Table 2 below.

TABLE 2
Composition of the vehicle for suspension
of particles as disclosed herein

| | | | |
|---|---|---|---|
| Sodium hyaluronate | 10 | mg | Active substance |
| Sodium chloride | 8.5 | mg | Isotonic agent |
| Sodium acid phosphate | 0.223 | mg | Buffering agent |
| Sodium dihydrogen | 40 | μg | Buffering agent |
| phosphate monohydrate | | | |

| | | |
|---|---|---|
| Hydrochloric acid | q.s. | For pH adjustment |
| Sodium hydroxide | q.s. | For pH adjustment |
| Water for injection | q.s. ad 1 mL | Solvent |

The microparticles are reconstituted by adding the vehicle to a vial with powder of microparticles to obtain a lenalidomide concentration of 33.3 mg/ml.

### Results

Suspensions of coated lenalidomide particles according to Example 1 and 2 and comparative Example 3, respectively are obtained.

### Example 10—In Vivo Pharmacokinetic Study

The present example describes a preclinical pharmacokinetic study of lenalidomide following administration of coated lenalidomide formulations according to the invention in male Sprague Dawley rats.

### Material and Method

Male Sprague Dawley rats weighing between 266 and 302 g at the day of administration are supplied by Charles River (UK). The animals are divided randomly into 4-8 animals per group according to Table 3. The duration of the study will be 14 days.

The intended administration area will be clipped free from hair prior to injection and the injection site will be marked. The suspensions described in Examples 1 and 2 and Comparative Examples 3-5 are drawn into a 1 mL BD syringe and single, subcutaneous injections (ca. 0.15 mL) will administered through a 20G needle (BD microlance) into the flank of each rat.

Alternatively, suspensions prepared essentially according to

Examples 1 and 2 and Comparative Examples 3-5, with the exception that they are sifted using a sonic sifter with 20 μm mesh, may be used in the study.

Administration will be performed no more than 10 minutes after preparation of the preparation of the formulations.

TABLE 3
Groups of animals included in the study.

| Group | Description |
|---|---|
| 1 | Uncoated particles in vehicle |
| | (Comparative Example 1) |
| 2 | Aluminium oxide coated particles in vehicle |
| | (Comparative Example 4) |
| 3 | Zinc oxide coated particles in vehicle |
| | (Comparative Example 5) |
| 4 | Coated particles according to Example 1 |
| 5 | Coated particles according to Example 2 |

Blood samples (ca 0.2 mL) will be collected from the tail vein into K₂EDTA tubes at the following time-points: 0.5, 1, 3, 6, 12, 24, 48, 72, 120, 168, 240, and 336 h post-dose. Actual sampling times will be recorded. As soon as practically possible following blood sampling, plasma will be separated by centrifugation (1500 g for 10 min at 4° C.). Animals will be sacrificed on the last day of the study. Plasma samples will be stored in a freezer at −80° C. pending analysis.

To determine the plasma concentration UPLC-MS/MS is used.

Samples are prepared by pipetting 25 μL of rat plasma into a 96 well plate, adding 25 μL 5% DMF in acetonitrile, 75 μL of an internal standard working solution using the TECAN Genesis liquid handling robot. The 96 well plates will be shaken for 15 minutes and centrifuged. All samples are then injected on a UPLC system with the set-up as specified in Table 1 above.

Pharmacokinetic analysis of lenalidomide in plasma is performed according to standard non-compartmental approach using Microsoft Excel for Mac (16.43, Microsoft, Redmond, Washington, USA). Dose-normalised plasma concentrations of lenalidomide after single subcutaneous administration of the various formulations (according to Examples 1-2 and Comparative Examples 3-5) is assessed and the following plasma pharmacokinetic parameters will be evaluated:
dose will be expressed in mg/kg body weight of the rat
‘tₘₐₓ’:the time to peak concentration expressed in hours
‘Cₘₐₓ’: the maximum concentration found in analysis expressed in μg/mL
‘tₗₐₛₜ’: the time of the last detectable concentration expressed in hours
‘t_{1/2,z}’: the terminal half-life expressed in hours ‘AUC_{∞}’: the area under concentration vs. time curve up to infinite time expressed in μg*h/mL
‘F’: the relative bioavailability expressed as a percentage ‘Cₘₐₓ/D’: the maximum concentration normalized to 1 mg/kg expressed in μg/mL/mg/kg body weight of the rat ‘AUCₗₐₛₜ/D’: the area under blood concentration vs. time curve up to the last detectable concentration normalized to 1 mg/kg expressed in μg*h/mL/mg/kg body weight of the rat
‘AUC_{∞}/D’: the area under concentration vs. time curve up to infinite time normalized to 1 mg/kg expressed in μg*h/mL/mg/kg body weight of the rat ‘Fr. Rel.₀₋₁₂ₕ’: the fraction released during the first twelve hours of the area under concentration vs. time curve up to infinite time expressed as a percentage.

### Results

It is expected that the groups administered coated lenalidomide particles according to the present invention will demonstrate a prolonged-release profile. In contrast, uncoated lenalidomide particles are expected to exhibit a rapid decline in plasma concentration following administration. Thus, advantageous plasma concentration-time profiles are expected to be observed for the formulations of the invention.

### Example 11—Transmission Electron Microscopy of Coated Particles

The present example aims at illustrating the structural features of particles coated using the method as disclosed herein. In particular, the Example illustrates the discrete layers which result from the deagglomeration step.

### Material and Method

Microparticles of indomethacin (Hangzhou APIChem Technology Co. Ltd., China) for coating were first prepared by wet ball milling (Fritsch, Premium line, Pulverisette 7, IDar-Oberstein, Germany) to give particles of a mean diameter of 5.9 μm. In order to measure the size of the particles obtained, 40 mg of the particles was placed in a test tube and 3 mL of solution of Tween-80 (0.5%; Merck, Kenilworth, NJ, USA) in water was added. The resultant suspension was gently vortexed (Vortex-Genie 2 (Scientific Industries Ltd., New York, USA)) for 1 minute and the particles size distribution was measured by means of laser diffraction (Shimadzu, SALD-7500nano, Kyoto, Japan).

The indomethacin particles were loaded into an ALD reactor (Picosun, SUNALE™ R-series, Espoo, Finland). 25 ALD cycles were performed at a reactor temperature of 50° C. Trimethyl aluminium and water were used as precursors, forming a first coating of aluminium oxide. The first layers was about 8 nm in thickness (as estimated from the number of ALD cycles with each cycle resulting in 0.3 nm).

The deagglomeration step was then carried out by removing the particles from the reactor and forcing them through a 100 μm mesh sieve, followed by a 20 μm mesh sieve. The deagglomerated powder was loaded back into the ALD reactor. A further series of 25 ALD cycles were performed, forming a second coating of aluminium oxide on top of the first. A second agitation step was then carried out in the same way as the first. The coating-deagglomeration steps were repeated twice further to form third and fourth separate coatings with the same thicknesses on the particles.

After the fourth coating step, particle size distribution was measured by means of laser diffraction (Shimadzu, SALD-7500nano, Kyoto, Japan) as described above. The average particle diameter of the coated particles was measured as about 6 μm.

The structure of the coating on the particles were next analyzed by transmission electron microscopy.

### Result

The transmission electron microscopy images obtained clearly showed a coating structure with discrete layer interfaces. The interfaces, which are not possible to characterize in terms of chemical composition, were found to correspond to the time when the coated particles were removed from the reactor and agitation/deagglomeration took place (see FIG. 4a) and b)). These layers can be described as similar to the rings in the cross-section of the trunk of a tree. The intermittent deagglomeration gives rise to a “signature” in any product that has been made using such a process, in which the outermost surface of the coated particles takes on a visibly different physical character compared to what lies beneath it.

Thus, the deagglomeration step gave rise to visible (by TEM) interfaces between discrete coating layers of metal oxides.

It is expected that a similar structure will be identifiable on lenalidomide microparticles comprising coatings as disclosed herein, such as particles according to Example 1 and 2. It is expected that the deagglomeration step used in the process of coating of said particles will give rise to similar structural features independent on what method is used to perform the deagglomeration.

### Example 12—Production of Coated Azacytidine Microparticles I, II and III and Particles Comprising Comparative Coating of Aluminium Oxide

The present example discloses the production of three types of azacytidine microparticles comprising a mixed oxide as disclosed herein. Additionally, microparticles comprising coatings of aluminum oxide were produced for comparison.

### Material and Method

Samples of microparticles of azacytidine (MSN Labs, India) were prepared by jet-milling. The particle size distribution, as determined by laser diffraction, was as follows: D 2.9 μm; D50 7.9 μm; D90 23.2 μm.

The powder was loaded to an ALD reactor and coating was performed as described in Example 1, however, deagglomeration was performed by means of sonic sifter (Tsutsui Sonic Agitated Sifting Machine SW-20AT) with a 20 μm mesh size sieve.

The obtained azacytidine particles, referred to herein as azacytidine particles I, had six layers of mixed oxide with a atomic ratio of approximately zinc:aluminium 3:1.

The same procedure as for azacytidine particles I was conducted to produced microparticles coated with mixed oxide coating comprising a atomic ratio of zinc:aluminium of 2:1, referred to as azacytidine particles II

The coating sequence was two ALD cycles employing diethyl zinc and water as precursors, followed by one cycle of trimethylaluminium and water, repeated ten times removal of the coated powder from the reactor, deagglomeration, reloading and repeating the same coating sequence, removal, deagglomeration until 4 sets of 30 cycles in total had been provided.

The same procedure as azacytidine particles I was conducted, but this time a thicker mixed oxide coating comprising a atomic ratio of zinc:aluminium of 3:1 was produced by conducting 6 sets of 24 ALD cycles. This resulted in particles referred to as azacytidine particles III.

The same microparticles of azacytidine (MSN Labs, India) were coated with a pure aluminium oxide coating. 7 ALD cycles were performed prior to removing the coated powder from the reactor and deagglomerated as described herein. The resultant deagglomerated powder was reloaded into the ALD reactor and subjected to a further 7 ALD cycles, followed by extraction, deagglomeration and reloading for 7 cycles was repeated twice thereafter, followed by reloading and subjecting to 2 times 14 cycles.

To determine the drug load (i.e. w/w % of azacytidine in the powder) of said particles, HPLC (Prominence-i (Shimadzu, Japan) equipped with a diode array detector (Shimadzu, Japan) set at 223 nm was employed using a 4.6×250 mm, 3 μm particles, C₁₈ column (Luna, Phenomenex, USA)). The nanoshell coatings were dissolved in 5 M phosphoric acid in DMSO and the slurry was diluted with DMSO, before filtration (0.2 μm RC, Lab Logistics Group, Germany) and further analyzed with HPLC (n=2).

### Result

Three types of microparticles of azacytidine coated with a mixed oxide as described herein were obtained as well as a microparticles of azacytidine coated with a comparative coating of aluminium oxide. The drug load was determined and was as follows: coated azacytidine microparticles I: 88.9%; coated azacytidine microparticles II: 86.6%; coated azacytidine microparticles III: 80.7%; and azacytidine microparticles with comparative aluminium oxide coating: 91.8%.

### Example 13—Preparation of Formulations of Coated Microparticles of Azacytidine

Coated microparticles of azacytidine according to Example 12 as well as uncoated azacytidine particles were prepared into formulations essentially as described in Example 9.

The formulation comprising uncoated azacytidine in Hyonate® vet exhited a concentration of azitidine of 10 mg/mL, which accords to 5 mg/kg body weight of a Sprague-Dawley rat.

The azacytidine microparticles with a comparative aluminium oxide coating were suspended Hyonate® vet to a achieve concentration of azacytidine in the formulation of 27 mg/mL, which accords to 13.5 mg/kg body weight of a Sprague-Dawley rat.

All three of the coated azacytidine microparticles I, II and III, exhibited the concentration of azacytidine in Hyonate® vet of 27 mg/mL, which accords to 13.5 mg/kg body weight of a Sprague-Dawley rat.

### Example 14—In Vivo Pharmacokinetic Study

The present example illustrates the expected effect on the pharmacokinetic properties of the coatings as described herein. It is expected that the effect will be similar for coated lenalidomide particles as the effect for the azatidine particles in this Example.

### Material and Method

Thirty-eight male Sprague Dawley rats weighing between 266 and 302 g at the day of administration were supplied by Charles River (UK). The animals were divided randomly into six animals per group.

The intended administration area was clipped free from hair prior to injection and the injection site was marked and administrations were performed as described in Example as summarized in Table 4. For groups 1-5, the particles were reconstituted according to Example 13, while group 6 PBS was used.

TABLE 4
Groups of animals according to the study.

| Group | Description | Dose |
|---|---|---|
| 1 | Uncoated particles | 5.0 ± 0.08 |
| 2 | Al₂O₃ coated particles | 13.5 ± 0.23 |
| 3 | (2:1) coated particles II | 13.6 ± 0.40 |
| 4 | (3:1) coated particles I | 13.5 ± 0.33 |
| 5 | (3:1) thicker coated particles III | 13.7 ± 0.17 |
| 6 | (3:1) thicker coated particles III in PBS | 13.7 ± 0.30 |

Blood samples were obtained and handled as described in Example 11.

Plasma concentration of azacytidine was determined with UPLC-MS/MS. Study samples were prepared by pipetting 25 μL of rat plasma into a 96 well plate, adding 25 μL 5% DMF in acetonitrile, 75 μL of an internal standard working solution using the TECAN Genesis liquid handling robot. The 96 well plates were then shaken for 15 minutes and centrifuged. All samples were then injected on a UPLC-MS/MS system. Separation was obtained with an ACQUITY BEH Amide Column, 2.5 μm, 2.1×100 mm, Waters at 25° C. using 10 mM ammonium formiate pH 3.4 in water with 0.125 μM Li Acetate as mobile phase A (MP A) and acetonitrile as mobile phase B (MP B).

Pharmacokinetic analysis of azacytidine in plasma was performed according to standard non-compartmental approach using Microsoft Excel for Mac (16.43, Microsoft, Redmond, Washington, USA). Maximum concentration, Cₘₐₓ, and related time, tₘₐₓ, were the coordinates of the highest concentration of the time course. tₗₐₛₜ was the time of the last detectable concentrations. The area under concentration vs. time curve up to the last detectable concentration, AUCₗₐₛₜ, was calculated using the linear trapezoidal rule.

The dose site on each animal was marked post-dosing and kept free from hair for the duration of the study. Dose site observations were performed at 24, 120, 168, and 336 hours post dosing.

### Results

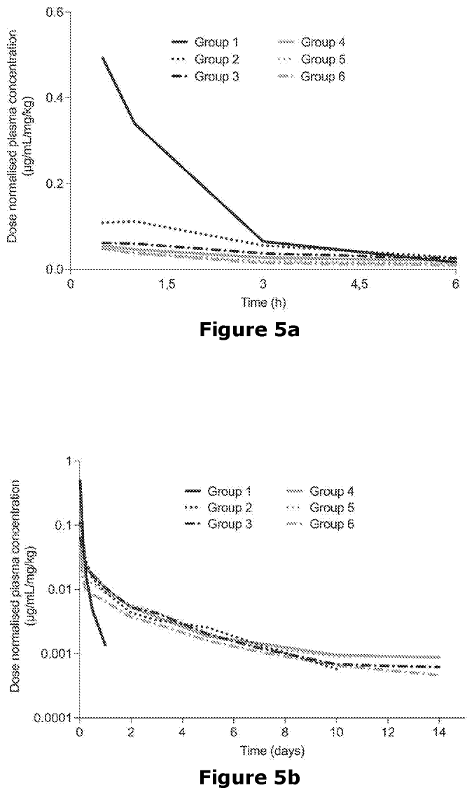

Dose-normalised plasma concentrations of azacytidine after single subcutaneous administration of the various formulations are presented in FIG. 5. The plasma pharmacokinetic parameters are also presented as mean values for the group of 6 rats (with standard deviations provided in parentheses) in Table 5 below

TABLE 5
Summary of pharmacokinetic parameters.

| Parameter | Group 1 | Group 2 | Group 3 | Group 4 | Group 5 | Group 6 |
|---|---|---|---|---|---|---|
| Dose | 5.0 | 13.5 | 13.6 | 13.5 | 13.7 | 13.7 |
| | (0.080) | (0.23) | (0.40) | (0.33) | (0.17) | (0.30) |
| tₘₐₓ | 0.50 | 0.83 | 0.75 | 0.50 | 0.50 | 0.50 |
| | (0.0) | (0.26) | (0.27) | (0.0) | (0.0) | (0.0) |
| Cₘₐₓ | 2.5 | 1.5 | 0.87 | 0.74 | 0.67 | 0.66 |
| | (0.11) | (0.096) | (0.071) | (0.055) | (0.096) | (0.11) |
| tₗₐₛₜ | 24 | 240 | 336 | 336 | 336 | 320 |
| | (0.0) | (0.0) | (0.0) | (0.0) | (0.0) | (39) |
| t_{1/2,z} | 1.6 | 58 | 65 | 140 | 102 | 77 |
| | (0.40) | (10) | (21) | (88) | (64) | (24) |
| AUC_{∞} | 4.9 | 17 | 17 | 18 | 16 | 11 |
| | (0.32) | (1.2) | (1.6) | (2.9) | (2.1) | (1.6) |
| F | 100 | 129 | 128 | 138 | 120 | 81 |
| | (5.0) | (12) | (9.0) | (21) | (15) | (11) |
| Cₘₐₓ/D | 0.49 | 0.11 | 0.064 | 0.055 | 0.049 | 0.048 |
| | (0.016) | (0.006) | (0.005) | (0.005) | (0.007) | (0.008) |
| AUCₗₐₛₜ/D | 0.98 | 1.2 | 1.2 | 1.2 | 1.0 | 0.75 |
| | (0.049) | (0.12) | (0.068) | (0.10) | (0.12) | (0.10) |
| AUC_{∞}/D | 0.98 | 1.3 | 1.3 | 1.4 | 1.2 | 0.80 |
| | (0.049) | (0.11) | (0.089) | (0.21) | (0.15) | (0.10) |
| Fr. Rel.₀₋₁₂ₕ | 96 | 39 | 29 | 22 | 19 | 22 |
| | (0.40) | (1.8) | (1.5) | (2.4) | (1.5) | (1.8) |

It can be seen that the plasma concentration profile was comparable for Groups 3 to 6, with maximal plasma concentration (Cₘₐₓ) reached within the first hour followed by a slow but steady decline in concentrations over the 14 day study period.

Group 2 had higher initial drug release compared with coating formulations having mixed oxides.

Comparing Group 5 to Group 6, it can be seen that the PBS-based vehicle resulted in a slightly lower AUC compared to when Hyonate vet was used, although the respective pharmacokinetic profiles were largely comparable.

Cₘₐₓ was lower for Groups 2 to 6 compared to uncoated azacytidine (Group 1), with approximately one fifth of the dose being released during the first day. When normalised for dose, the difference is about an order of magnitude. Also, the residual area (correlating to unreleased drug) following the last sampling time (336 hours post-dosing) was <12%.

The results for Group 2, showed a slightly different profile characterized by a larger fraction of the dose released during the first day, a higher Cₘₐₓ, and a shorter duration.

In summary, Groups 3-6 demonstrated a prolonged-release profile, which differs from the rapid decline following administration of uncoated azacytidine. Similar, advantageous plasma concentration-time profiles were observed for all of the formulations comprising coatings of mixed oxides.

It is noteworthy that the coating comprising aluminium oxide alone (group 2) did not achieve a desirable pharmacokinetic profile. It is expected that similar results will be observed for coated lenalidomide particles in Example 11.

### Comparative Example 15

Coated Lenalidomide Microparticles Using Continuous Flow ALD Process I Microparticles of lenalidomide 99.9% (APIChem, China) were used as received. The mean diameter of the lenalidomide particles was 10 μm as determined by laser diffraction (Shimadzu, SALD-7500nano, Kyoto, Japan). The particle size distribution, as determined by laser diffraction, was as follows: D₁₀ 2.7 μm; D₅₀ 10.4 μm and D₉₀ 24.9 μm.

The microparticles were coated as described in steps 1-4.
1. The microparticles were loaded into an ALD reactor (Picosun, SUNALE™ R-series, Espoo, Finland) and subjected to three ALD cycles employing diethyl zinc and water as precursors, followed by one ALD cycle of trimethylaluminium and water as precursors at a reactor temperature of 50° C. This was repeated six times, i.e. 6×(3 cycles of Zn+1 cycle of Al), resulting in a total of 24 cycles. By this process, a first layer of mixed oxide with a atomic ratio of approximately 3:1 zinc:aluminium was formed.

The ALD reactor comprised a reaction chamber into which the microparticles were loaded. The ALD reactor further comprised precursor bottles containing each precursor separately, each precursor bottle being coupled to the reaction chamber via a valve. The ALD reactor also comprised a pump and associated piping for pumping an inert gas such as nitrogen through the reaction chamber, which pump was also coupled to the reaction chamber via a valve.

The ALD-cycle was performed as follows, wherein steps a to e represent a first cycle, subsequent cycles start from step d as specified in step I and the last cycle ends at step k:
a. Reagent pulse: Water was evaporated and carried into the reaction chamber by inert nitrogen gas by opening a valve to the precursor bottle for 0.1 s. Water was adsorbed to the surface of the drug particles, presenting hydroxyl groups on the exterior or the particle.
b. The reactor was thereafter pumped for 3 s.
c. Steps a-b above were repeated 100 times.
d. Purging pulse: The chamber was purged with nitrogen. Gaseous water, and organic gases in case this is not the first cycle, was removed.
e. Reagent pulse: diethylzinc or trimethylaluminum was evaporated and carried into the reaction chamber by inert nitrogen gas by opening a valve to the precursor bottle for 0.1 s. Diethylzinc or trimethylaluminum was adsorbed to the surface of the drug particles and reacts with hydroxyl groups. This releases ethane from diethylzinc or methane from trimethylaluminum.
f. The reactor was thereafter pumped for 3 s.
g. Steps e-f above were repeated 100 times.
h. Purging pulse: The chamber was purged with nitrogen. Non-reacted reagents and organic gases were removed.
i. Reagent pulse: Water was evaporated and carried into the reaction chamber by inert nitrogen gas by opening a valve to the precursor bottle for 0.1 s. Water was adsorbed to the surface of the drug particles and reacted with the metalloorganic surface. Remaining ethyl or methyl groups are converted to ethane and methane, respectively. The surface was thereby covered in a metal oxide layer, presenting hydroxyl groups on the exterior of the particle.
j. The reactor was thereafter pumped for 3 s.
k. Steps i-j above was repeated 100 times
l. The cycle was repeated from step d.
2. Next the powder was removed from the reactor and deagglomerated by means of sonic sifter (Tsutsui Sonic Agitated Sifting Machine SW-20AT) with a 32 μm mesh size sieve.
3. The resultant deagglomerated powder was re-loaded into the ALD reactor and steps 1-2 were repeated two times, forming a second and a third layer of mixed oxide with a atomic ratio of approximately 3:1 zinc:aluminium.
4. The resultant deagglomerated powder was re-loaded into the ALD reactor and step 1 was repeated one time.

### Comparative Example 16

### Coated Lenalidomide Microparticles Using Continuous Flow ALD Process II

This was similar to Comparative Example 15 except that two additional layers were formed. In other words, step 3 was repeated once before step 4 was performed.

### Example 17

### Coated Lenalidomide Microparticles Using Stop-Flow ALD Process I

Microparticles of lenalidomide 99.9% (Kyongbo, South Korea) were used as received. The mean diameter was determined as described in Example 15 above. The particle size distribution, as determined by laser diffraction, was as follows: D₁₀ 0.6 μm; D₅₀ 3.9 μm and D₉₀ 15.6 μm.

The microparticles were coated essentially as described in steps 1-4 of Comparative Example 15, except that the ALD-cycle was performed as follows (steps a-e represent the first cycle, subsequent cycles start from step a as specified in step e.
a. Reagent pulse 1:
   i. The valve on the piping between the pump and the ALD reactor was closed.
   ii. The valve on the water precursor bottle was opened for 1 s letting evaporated water fill the reaction chamber.
   iii. The valve to the water precursor bottle was closed and, before opening the pump valve again, the chamber was rested for 30 s (soaking time) to ensure the water vapor adsorbed to the surface of the drug particles, presenting hydroxyl groups on the exterior or the particles.
   iv. The reactor was thereafter pumped for 9 s.
   v. Steps i-iv above were repeated 20 times.
b. Purging pulse: The chamber was purged with nitrogen in a continuous flow. Gaseous water, and organic gases in case this is not the first cycle, was removed.
c. Reagent pulse:
   i. The valve on the piping between the pump and the ALD reactor was closed.
   ii. The valve on the diethylzinc or trimethylaluminium precursor bottle was opened for 1 s letting evaporated metal containing precursor fill the reaction chamber.
   iii. The valve to the precursor bottle was closed and, before opening to the pump again, the chamber was rested for 30 s (soaking time) to ensure the metal containing precursor vapor reacted with the hydroxyl groups on the surface of the drug particles.
   iv. The reactor was thereafter pumped for 9 s.
   v. Steps a-d above were repeated 20 times.
d. Purging pulse: The chamber was purged with nitrogen in a continuous flow. Non-reacted reagents and organic gases were removed.
e. The cycle was repeated from step a.

### Example 18

### Coated Lenalidomide Microparticles Using Stop-Flow ALD Process II

This was similar to Comparative Example 17 but two additional layers were formed. In other words, step 3 was repeated once before step 4 was performed.

### Example 19

### Determination of Drug Load

The present example describes the analysis of the drug load of lenalidomide of the particles obtained in Comparative Examples 15 and 16 and Examples 17 and 18.

### Material and Method

To determine the drug load (i.e. w/w % of lenalidomide in the powder), UPLC (Prominence-i (Shimadzu, Japan) equipped with a diode array detector (Shimadzu, Japan) set at 223 nm was employed using a 4.6×100 mm, 2.6 μm particles, C₁₈WP column (SunShell, ChromaNik Technologies Inc, Osaka, Japan)) was used. The materials were dissolved in 2 M phosphoric acid in acetonitrile/water (1:1) and was diluted with methanol/12.5 mM phosphate buffer pH 3.4 (4:1) before filtration (0.2 μm RC, Lab Logistics Group, Germany) and further analyzed with UPLC (n=2).

The UPLC assay is set-up according to Table 6.

TABLE 6
Parameters for the UPLC-method.

| Column | SunShell C18WP 4.6*100 mm, | |
|---|---|---|
| | 2.6 μm particles, Nanexa column ID 1. | |
| Temperature, column oven | 20° C. | |
| Flow | 0.7 mL/min | |
| Gradient program | Time (min) | % Mobile phase B |
| | 0-0.5 | 10 |
| | 4 | 12 |
| | 12 | 32 |
| | 15 | 32 |
| | 17.50 | 10 |
| | 17.50-22 | 10 |

| | |
|---|---|
| Mobile phase A | 5 mM phosphate buffer pH 3.4 |
| Mobile phase B | Methanol |
| Wash solution | 50% Acetonitrile in water |
| Run time | 22 min |
| Temperature, autosampler | 25° C. |
| Injection volume | 5 μL |
| UV detection wavelength | 223 nm |

Concentrations of injected samples were calculated automatically by the software supplied by the manufacturer. Drug load was calculated according to the equation below, wherein C_{LEN} is the analyzed concentration of lenalidomide and mₛₐₘₚₗₑ is sample mass:

$$Drugload ( \% ) = 100 \times \frac{{C}_{LEN} ( mg / L )}{\frac{{m}_{sample} ( mg )}{End Volume ( L )}}$$

### Results

The drug load lenalidomide of the particles according to Comparative Example 15 was determined as 94.8%.

The drug load lenalidomide of the particles according to Comparative Example 16 was determined as 92.6%.

The drug load lenalidomide of the particles according to Example 17 was determined as 76.0%.

The drug load lenalidomide of the particles according to Example 18 was determined as 67.7%.

### Example 20

### Coating Integrity

The present example describes the analysis of the coating integrity of the particles obtained in Comparative Examples 15 and 16 and Examples 17 and 18.

The coating integrity of the coated lenalidomide was determined by preparing a suspension of the coated material in DMSO, a solvent which dissolves the API but not the coating material. Therefore, release of lenalidomide from the product can only happen due to ‘defects’ in coating. By measuring the released lenalidomide with an HPLC method, the integrity of the coating can be assessed. The lower the percentage of the released lenalidomide the better the coating integrity.

### Material and Method

To determine the coating integrity (i.e. w/w % of lenalidomide released in the powder), UPLC (Prominence-i (Shimadzu, Japan) equipped with a diode array detector (Shimadzu, Japan) set at 223 nm was employed using a 4.6×100 mm, 2.6 μm particles, C18WP column (SunShell, ChromaNik Technologies Inc, Osaka, Japan)) was used. The 25 mg of the materials were dispersed in 25 mL DMSO and put on a turning table for 3 h. A 1 mL sample was withdrawn for filtration (0.2 μm RC, Lab Logistics Group, Germany) and further analysis with UPLC (n=1) according to set-up in Table 6 above.

Concentrations of injected samples were calculated automatically by the software supplied by the manufacturer. The amount API dissolved in the coating integrity assay was calculated according to the equation below, wherein ca is the concentration of API from analysis (mg/mL), Vₜ is the total sample volume, (m@), m_{API} is the mass of API weighed in (mg), and drugload is the API content of the coated API (fraction):

$$Amount API dissolved ( \% ) = 100 \cdot \frac{{c}_{a} \cdot {V}_{t}}{{m}_{API} \cdot drugload}$$

### Results

The amount API dissolved in the coating integrity assay of the particles according to Comparative Example 15 was determined as 74%.

The amount API dissolved in the coating integrity assay of the particles according to Comparative Example 16 was determined as 51%.

The amount API dissolved in the coating integrity assay of the particles according to Example 17 was determined as 15%.

The amount API dissolved in the coating integrity assay of the particles according to Example 18 was determined as 7%.

### Example 21

### Preparation of Formulations

The present example describes the preparation of formulations of the coated lenalidomide particles as disclosed herein.

### Material and Method

Suspensions of coated microparticles of lenalidomide according to Comparative Examples 15 and 16 and Examples 17 and 18 was prepared.

The powder of coated microparticles of lenalidomide according to Comparative Examples 15 and 16 and Examples 17 and 18 was mixed together with Hyonate® vet (Boehringer Ingelheim Animal Health, France) as vehicle. Hyonate® vet is a veterinary medicinal product used in injecting animals comprising a sterile, isotonic, phosphate buffered solution of 10 mg/mL of sodium hyaluronate (pH 7.4). The composition of Hyonate® vet is a presented in Table 7 below.

TABLE 7
Composition of the vehicle for suspension
of particles as disclosed herein.

| | | | |
|---|---|---|---|
| Sodium hyaluronate | 10 | mg | Active substance |
| Sodium chloride | 8.5 | mg | Isotonic agent |
| Sodium acid phosphate | 0.223 | mg | Buffering agent |
| Sodium dihydrogen | 40 | μg | Buffering agent |
| phosphate monohydrate | | | |

| | | |
|---|---|---|
| Hydrochloric acid | q.s. | For pH adjustment |
| Sodium hydroxide | q.s. | For pH adjustment |
| Water for injection | q.s. ad 1 mL | Solvent |

The microparticles were reconstituted by adding the vehicle to a vial with powder of microparticles to obtain a lenalidomide concentration of:
50 mg/mL lenalidomide of the particles according to Comparative Example 15,
5 mg/mL and 50 mg/ml lenalidomide of the particles according to Comparative Example 16,
20 mg/mL lenalidomide of the particles according to Example 17, and
20 mg/mL lenalidomide of the particles according to Example 18.

### Results

Suspensions of coated lenalidomide particles according to Comparative Examples 15 and 16 and Examples 17 and 18 respectively, were obtained.

### Comparative Example 22

### In Vivo Pharmacokinetic Study I

The present example describes a preclinical pharmacokinetic study of lenalidomide following administration of coated lenalidomide formulations according to Comparative Examples 15 and 16 in male and female Sprague Dawley rats.

### Material and Method

Male and Female Sprague Dawley rats weighing between 231 and 285 g at the day of administration were supplied by Charles River (UK). The duration of the study was 14 days. 36 rats were used in the study.

The administration areas were clipped free from hair prior to injection and the injection site will be marked. A suspension prepared as described in Example 21 with coated lenalidomide particles according to Comparative Example 15 and 16 were drawn into a 1 mL BD syringe and single, subcutaneous injections (ca. 0.12 mL) was administered through a 23G needle (BD microlance) into the flank of each rat.

Blood samples (ca 0.2 mL) were be collected from the tail vein into K₂EDTA tubes at the following time-points: 0.25, 0.5, 1, 2, 3, 6, 12, 24, 48, 72, 120, 168, 264, and 336 h post-dose. As soon as practically possible following blood sampling, plasma was separated by centrifugation (1500 g for 10 min at 4° C.). Animals were sacrificed on the last day of the study. Plasma samples were stored in a freezer at −80° C. pending analysis.

To determine the plasma concentration, UPLC-MS/MS was used.

Samples were prepared by pipetting 35 μL of rat plasma into a 96 well plate, adding 35 μL 5% DMF in acetonitrile, 70 μL of an internal standard working solution using the TECAN Genesis liquid handling robot. The 96 well plates were shaken for 15 minutes and centrifuged. All samples were then injected on a UPLC-MS/MS system (Xevo TQ-s micro coupled to an Acquity I-Class UPLC system, Waters, Milford, MA, USA) with the set-up as specified in Table 8 below.

TABLE 8
Parameters for the UPLC-MS/MS-method.

| | |
|---|---|
| Column | Acquity CSH C18 (50 × 2.1 mm, 1.7 μm) |
| Mobile phase A | 0.10% Formic Acid in Water |
| Mobile phase B | ACN:DMSO (95:5) |
| Wash solution | ACN:MeOH (1:1) |

Pharmacokinetic analysis of lenalidomide in plasma was performed according to standard non-compartmental approach using PKanalix (2021, Lixoft, Antony, France). Dose-normalised plasma concentrations of lenalidomide after single subcutaneous administration of the formulation was assessed and the following plasma pharmacokinetic parameters was evaluated:
Dose will be expressed in mg/kg body weight of the rat.
‘Cₘₐₓ’: the maximum concentration found in analysis expressed in ng/mL.
‘AUC_{∞}’: the area under concentration vs. time curve up to infinite time expressed in ng*h/mL.
‘Cₘₐₓ/D’: the maximum concentration normalized to 1 mg/kg expressed in ng/mL/mg/kg body weight of the rat.
‘AUC_{∞}/D’: the area under concentration vs. time curve up to infinite time normalized to 1 mg/kg expressed in ng*h/mL/mg/kg body weight of the rat.
‘Fr. Rel.₀₋₁₂ₕ’: the fraction released during the first twelve hours of the area under concentration vs. time curve up to infinite time expressed as a percentage.

### Results

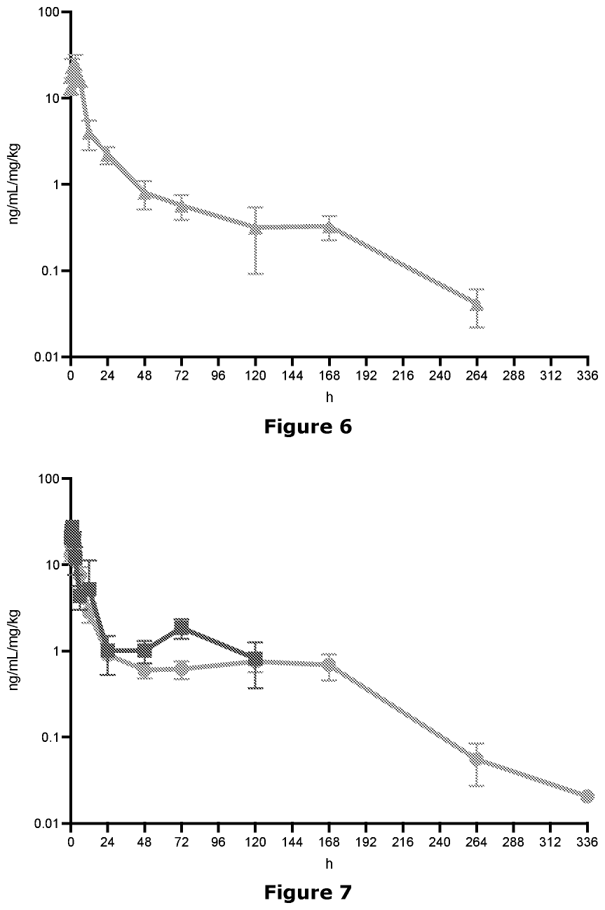

FIGS. 6 and 7 and Table 9 show the respective plasma concentration time curves (ng/mL plasma concentration lenalidomide versus h sampling time) for samples obtained by Comparative Examples 15 and 16, respectively.

TABLE 9
Results of plasma pharmacokinetic parameters evaluation.

| | Comparative | Comparative | Comparative |
|---|---|---|---|
| Parameter | Example 1 | Example 2 | Example 2 |
| Dose | 50 | 5 | 50 |
| Cₘₐₓ | 1250 | 142 | 986 |
| AUC_{∞} | 17300 | 712 | 14000 |
| AUC_{0-12 h} | 12000 | 650 | 6974 |
| Cₘₐₓ/D | 25.0 | 28.4 | 19.7 |
| AUC_{∞}/D | 346 | 142 | 280 |
| AUC_{0-12 h}/D | 240 | 130 | 139 |
| Fr. Rel._{0-12 h} | 0.694 | 0.913 | 0.498 |

### Example 23

### In Vivo Pharmacokinetic Study II

The present example describes a preclinical pharmacokinetic study of lenalidomide following administration of coated lenalidomide formulations according to Examples 17 and 18 in male and female Sprague Dawley rats.

Essentially the same method as that described in Example 22 above was conducted on male Sprague Dawley rats weighing between 260 and 343 g (Charles River, UK). The duration of the study was 14 days. 44 rats were used in the study, with ca. 0.15 mL subcutaneous injections.

### Results

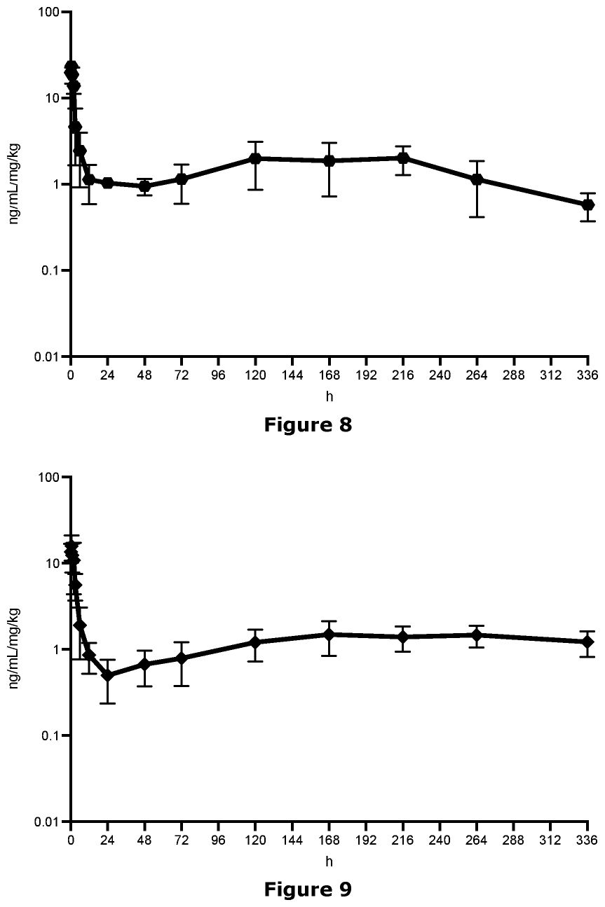

FIGS. 7 and 8 and Table 10 show the respective plasma concentration time curves (ng/mL plasma concentration lenalidomide versus h sampling time) for samples obtained by Examples 17 and 18, respectively.

TABLE 10
Results of plasma pharmacokinetic parameters evaluation.

| | Comparative | Comparative |
|---|---|---|
| Example | Example 3 | Example 4 |
| Dose | 10 | 10 |
| Cₘₐₓ | 239 | 152 |
| AUC_{∞} | 7693 | 9279 |
| AUC_{0-12 h} | 668 | 452 |
| Cₘₐₓ/D | 23.9 | 15.2 |
| AUC_{∞}/D | 769.3 | 927.9 |
| AUC_{0-12 h}/D | 66.8 | 45.2 |
| Fr. Rel._{0-12 h} | 0.087 | 0.049 |

The dose normalized maximum concentration was lower for Example 17 compared with Comparative Example 15 indicated by both a lower maximum concentration and a lower fraction released during the first twelve hours.

The dose normalized maximum concentration was lower for Example 18 compared with Comparative Example 16 indicated by both a lower maximum concentration and a lower fraction released during the first twelve hours.

Altogether, Comparative Example 22 and Example 23 demonstrated that the coated lenalidomide particles produced by stop-flow ALD showed a release profile with lower initial release than the coated lenalidomide particles produced using continuous flow ALD.

1. A plurality of coated particles useful in the treatment of hematologic cancers, such as multiple myeloma, said coated particles comprising:
(a) a solid core comprising a compound selected from the group consisting of lenalidomide and pomalidomide, or a pharmaceutically acceptable salt thereof, said core having a mean diameter of the between 0.1 μm and 50 μm; and
(b) a coating surrounding, enclosing and/or encapsulating said core, wherein the coating comprises a mixed oxide of at least two metal or metalloid oxides selected from the group consisting of iron oxide, zinc oxide, silicon oxide, titanium oxide, and aluminium oxide.

2. The plurality of coated particles according to claim 1, wherein said compound is lenalidomide.

3. The plurality of coated particles according to claim 1 or 2, wherein said coating comprises 1 to 20 discrete layers, such as 3 to 7 discrete layers, such as 4 to 6 discrete layers, wherein each layer comprises at least one oxide selected from the group consisting of iron oxide, zinc oxide, silicon oxide, titanium oxide, and aluminium oxide, and wherein at least one out of said 1 to 20 discrete layers comprises said mixed oxide.

4. The plurality of coated particles according to claim 3, wherein at least 2, such as at least 3, such as at least 4, such as all of said discrete layers comprise said mixed oxide.

5. The plurality of coated particles according to claim 3 or 4, wherein said at least one oxide is selected from the group consisting of zinc oxide, silicon oxide, and aluminium oxide, such as zinc oxide or aluminium oxide.

6. The plurality of coated particles according to any one of claims 1 to 5, wherein said at least two metal and/or metalloid oxides in said mixed oxide are selected from the group consisting of zinc oxide, silicon oxide, and aluminium oxide, such as zinc oxide and silicon oxide.

7. The plurality of coated particles according to any one of claims 1 to 6, wherein said at least two metal and/or metalloid oxides in said mixed oxide are zinc oxide and aluminium oxide.

8. The plurality of coated particles according to claim 7, wherein the atomic ratio of Zn to Al is between 1 to 1 and 6 to 1, such as between 2 to 1 and 5 to 1, such as 2 to 1 and 4 to 1, such as 3 to 1.

9. The plurality of coated particles according to any one of the preceding claims, which particles:
(a) have a weight-, number-, or volume-based mean diameter that is between 10 nm (such as about 0.1 μm) and about 700 μm (e.g. about 100 or about 50 μm, or one or more of the particle size ranges described herein); and
(b) comprise solid cores comprising a compound selected from the group consisting of lenalidomide and pomalidomide, or a pharmaceutically-acceptable salt of either compound, coated, at least in part, by a coating of inorganic material comprising mixture of:
(i) zinc oxide; and
(ii) one or more other metal and/or metalloid oxides,
wherein the atomic ratio ((i):(ii)) is at least about 1:6 (e.g. about 1:1) and up to and including about 6:1.

10. The plurality of coated particles according to claim 9, wherein the ratio of zinc oxide to other metal and/or metalloid oxides is between about 2:1 and about 5:1.

11. The plurality of coated particles according to any one of the preceding claims, wherein more than one discrete layer of the mixture of oxides is applied to the core sequentially.

12. The plurality of coated particles according to claim 5, wherein between 3 and 10 discrete layers of the mixture of oxides are applied.

13. A method of preparing a plurality of coated particles according to any one of claims 1 to 12, wherein the coated particles are made by applying the precursors of at least two metal and/or metal oxides forming a mixed oxide on the solid cores, and/or the previously-coated solid cores, by a gas phase deposition technique, such as atomic layer deposition.

14. A method of according to claim 13, said method comprising the steps of:
(1) applying precursors of one or more metal and/or metalloid oxides selected from the group consisting of iron oxide, zinc oxide, silicon oxide, titanium oxide, and aluminium oxide, whereby a layer is formed on said solid cores by way of a gas phase deposition technique;
(2) disaggregating the coated solid core aggregates formed during step (1);
(3) applying precursors of at least one metal and/or metalloid oxides selected from the group consisting of iron oxide, zinc oxide, silicon oxide, titanium oxide, and aluminium oxide to the disaggregated, coated solid cores from step (2) whereby another layer is formed on said previously-coated solid cores by way of a gas phase deposition technique; and
(4) repeating steps (2) and (3) one or more times to increase the total thickness of the coating that encloses said solid core,
wherein at least one of the layers is formed by applying precursors of at least two metal and/or metalloid oxides selected from the group consisting of iron oxide, zinc oxide, silicon oxide, titanium oxide, and aluminium oxide, whereby a mixed oxide is formed.

15. The method according to claim 14, wherein said disaggregating coated particles aggregates is done by way of a mechanical sieving technique.

16. The method according to claim 15, wherein the mechanical sieving technique comprises sonic sifting or vibrational sieving.

17. The method according to any one of claims 14 to 16, wherein step 2 further comprises a step of discharging the coated solid cores from the gas phase deposition reactor, prior to disaggregating coated solid core aggregates formed during step (1); and the step of charging the coated solid cores back to the reactor subsequent to disaggregating coated solid cores aggregates formed during step (1).

18. A pharmaceutical or veterinary composition, comprising plurality of coated particles according to any one of claims 1 to 12 and a pharmaceutically acceptable diluent, carrier and/or excipient.

19. A composition according to claim 18 in the form of a sterile injectable and/or infusible dosage form.

20. The composition according to claim 19 in the form of a liquid, a sol, or a gel, administrable via a surgical administration apparatus or a syringe, which liquid, sol, or gel forms a subcutaneous depot formulation.

21. A plurality of coated particles according to any one of claims 1 to 12 or a pharmaceutical composition according to any one of claims 18 to 20 for use in the treatment of a hematologic cancer.

22. The use of a plurality of coated particles according to any one of claims 1 to 12 or a pharmaceutical composition according to any one of claims 18 to 20 for the manufacture of a medicament for the treatment of a hematologic cancer.

23. A method of treatment of a hematologic cancer, which method comprises the administration of a plurality of coated particles according to any one of claims 1 to 12 or a pharmaceutical composition according to any one of claims 18 to 20 to a patient in need of such treatment.

24. A plurality of coated particles for use or a pharmaceutical composition for use according to claim 21, a use according to claim 22, or a method according to claim 23, wherein the hematologic cancer is multiple myeloma.
